SEQUENCE LISTING (86 sequences)

SEQ ID NO: 1 - DNA, 20 bp, synthetic construct
  source 1..20: mol_type = other DNA
tctctattcttttgcgccgg

SEQ ID NO: 2 - DNA, 18 bp, synthetic construct
  source 1..18: mol_type = other DNA
cttggacagtgggcgcac

SEQ ID NO: 3 - DNA, 21 bp, synthetic construct
  source 1..21: mol_type = other DNA
aggaagctcgcggaccagtac

SEQ ID NO: 4 - DNA, 22 bp, synthetic construct
  source 1..22: mol_type = other DNA
ggtggtccttcttgtgctgcac

SEQ ID NO: 5 - DNA, 22 bp, synthetic construct
  source 1..22: mol_type = other DNA
acgaggcttcttctacacaccc

SEQ ID NO: 6 - DNA, 22 bp, synthetic construct
  source 1..22: mol_type = other DNA
tccacaatgccacgcttctgca

SEQ ID NO: 7 - DNA, 22 bp, synthetic construct
  source 1..22: mol_type = other DNA
gaagtctaccaaagctcacgcg

SEQ ID NO: 8 - DNA, 22 bp, synthetic construct
  source 1..22: mol_type = other DNA
ggaactccttctccagctctag

SEQ ID NO: 9 - DNA, 22 bp, synthetic construct
  source 1..22: mol_type = other DNA
agctagaggtgaagatccgcga

SEQ ID NO: 10 - DNA, 22 bp, synthetic construct
  source 1..22: mol_type = other DNA
gcaggacaatcctggagttctc

SEQ ID NO: 11 - DNA, 23 bp, synthetic construct
  source 1..23: mol_type = other DNA
gataatgggagcaaccaagtggc

SEQ ID NO: 12 - DNA, 22 bp, synthetic construct
  source 1..22: mol_type = other DNA
cagtatgtgccagcaggaagac

SEQ ID NO: 13 - DNA, 20 bp, synthetic construct
  source 1..20: mol_type = other DNA
acgcttcttctggaagacca

SEQ ID NO: 14 - DNA, 20 bp, synthetic construct
  source 1..20: mol_type = other DNA
cggtcaagaaaagccttcac

SEQ ID NO: 15 - DNA, 22 bp, synthetic construct
  source 1..22: mol_type = other DNA
gcttcagcaaggaggaggtcat

SEQ ID NO: 16 - DNA, 22 bp, synthetic construct
  source 1..22: mol_type = other DNA
tctggagttggcacttctcgct

SEQ ID NO: 17 - DNA, 23 bp, synthetic construct
  source 1..23: mol_type = other DNA
cctattcgttggggatgacagag

SEQ ID NO: 18 - DNA, 22 bp, synthetic construct
  source 1..22: mol_type = other DNA
tctgtctccgagtcctgcttct

SEQ ID NO: 19 - DNA, 22 bp, synthetic construct
  source 1..22: mol_type = other DNA
gctgaaggacagcgagaagatc

SEQ ID NO: 20 - DNA, 23 bp, synthetic construct
  source 1..23: mol_type = other DNA
gggtccattttgggctttttccg

SEQ ID NO: 21 - DNA, 22 bp, synthetic construct
  source 1..22: mol_type = other DNA
gaaagagtggcaacctgccttc

SEQ ID NO: 22 - DNA, 24 bp, synthetic construct
  source 1..24: mol_type = other DNA
gcaccaagttttactacatctgcc

SEQ ID NO: 23 - DNA, 19 bp, synthetic construct
  source 1..19: mol_type = other DNA
gcagaggagtgtccggcag

SEQ ID NO: 24 - DNA, 22 bp, synthetic construct
  source 1..22: mol_type = other DNA
ggattcccattggtgtctggaa

SEQ ID NO: 25 - DNA, 21 bp, synthetic construct
  source 1..21: mol_type = other DNA
ctgcctagagctccctctttc

SEQ ID NO: 26 - DNA, 20 bp, synthetic construct
  source 1..20: mol_type = other DNA
gcctgtccaacagaagggtc

SEQ ID NO: 27 - DNA, 20 bp, synthetic construct
  source 1..20: mol_type = other DNA
cagccacccgagattgagca

SEQ ID NO: 28 - DNA, 20 bp, synthetic construct
  source 1..20: mol_type = other DNA
tagtagcgacgggcggtgtg

SEQ ID NO: 29 - DNA, 22 bp, synthetic construct
  source 1..22: mol_type = other DNA
acaaaagccgctcgcaagagtg

SEQ ID NO: 30 - DNA, 22 bp, synthetic construct
  source 1..22: mol_type = other DNA
tttctcgcaccagacgctggaa

SEQ ID NO: 31 - DNA, 20 bp, synthetic construct
  source 1..20: mol_type = other DNA
gagatggggaagatgctggg

SEQ ID NO: 32 - DNA, 20 bp, synthetic construct
  source 1..20: mol_type = other DNA
ggaggacacagtcagggaga

SEQ ID NO: 33 - DNA, 25 bp, synthetic construct
  source 1..25: mol_type = other DNA
tccttttcatgcaacacccaacact

SEQ ID NO: 34 - DNA, 25 bp, synthetic construct
  source 1..25: mol_type = other DNA
tccaaatgctggtaacactgtggtc

SEQ ID NO: 35 - DNA, 20 bp, synthetic construct
  source 1..20: mol_type = other DNA
gcagtgtgccaaaccagatg

SEQ ID NO: 36 - DNA, 20 bp, synthetic construct
  source 1..20: mol_type = other DNA
gggtttgccggggtacaata

SEQ ID NO: 37 - DNA, 20 bp, synthetic construct
  source 1..20: mol_type = other DNA
attctgcaggggccgagcct

SEQ ID NO: 38 - DNA, 20 bp, synthetic construct
  source 1..20: mol_type = other DNA
atcacacggagccgcttggc

SEQ ID NO: 39 - DNA, 20 bp, synthetic construct
  source 1..20: mol_type = other DNA
gctcatggctgaagatgagg

SEQ ID NO: 40 - DNA, 19 bp, synthetic construct
  source 1..19: mol_type = other DNA
caggcgcttgtccttcttc

SEQ ID NO: 41 - DNA, 24 bp, synthetic construct
  source 1..24: mol_type = other DNA
acgtttctgcaaagctgtctagtt

SEQ ID NO: 42 - DNA, 24 bp, synthetic construct
  source 1..24: mol_type = other DNA
ggcttcaaaccattcagtaacttc

SEQ ID NO: 43 - DNA, 24 bp, synthetic construct
  source 1..24: mol_type = other DNA
tggctgtgaacaaacttcataaat

SEQ ID NO: 44 - DNA, 24 bp, synthetic construct
  source 1..24: mol_type = other DNA
caccgtggcttaaaagtttctatt

SEQ ID NO: 45 - DNA, 24 bp, synthetic construct
  source 1..24: mol_type = other DNA
ttgctcctagcctatctttcctta

SEQ ID NO: 46 - DNA, 24 bp, synthetic construct
  source 1..24: mol_type = other DNA
ctttagaattcctggacccttctc

SEQ ID NO: 47 - DNA, 20 bp, synthetic construct
  source 1..20: mol_type = other DNA
cttctggtcgccaagttcag

SEQ ID NO: 48 - DNA, 20 bp, synthetic construct
  source 1..20: mol_type = other DNA
agcagataaagcgtgccaag

SEQ ID NO: 49 - DNA, 24 bp, synthetic construct
  source 1..24: mol_type = other DNA
gggaacatagagaaagaggtctca

SEQ ID NO: 50 - DNA, 24 bp, synthetic construct
  source 1..24: mol_type = other DNA
aattaatctcagcttccccctaac

SEQ ID NO: 51 - DNA, 24 bp, synthetic construct
  source 1..24: mol_type = other DNA
tttatagtctcagagcccatctcc

SEQ ID NO: 52 - DNA, 24 bp, synthetic construct
  source 1..24: mol_type = other DNA
cccctggttaagactctaatgacc

SEQ ID NO: 53 - DNA, 22 bp, synthetic construct
  source 1..22: mol_type = other DNA
gacctcatcttccgtgtgatct

SEQ ID NO: 54 - DNA, 22 bp, synthetic construct
  source 1..22: mol_type = other DNA
cagatgacactatgggggtgat

SEQ ID NO: 55 - DNA, 22 bp, synthetic construct
  source 1..22: mol_type = other DNA
acaaaagccgctcgcaagagtg

SEQ ID NO: 56 - DNA, 22 bp, synthetic construct
  source 1..22: mol_type = other DNA
tttctcgcaccagacgctggaa

SEQ ID NO: 57 - DNA, 21 bp, synthetic construct
  source 1..21: mol_type = other DNA
gcagcctttgtgaaccaacac

SEQ ID NO: 58 - DNA, 20 bp, synthetic construct
  source 1..20: mol_type = other DNA
ccccgcacactaggtagaga

SEQ ID NO: 59 - DNA, 21 bp, synthetic construct
  source 1..21: mol_type = other DNA
ctaagagcgagttggcactga

SEQ ID NO: 60 - DNA, 21 bp, synthetic construct
  source 1..21: mol_type = other DNA
gaggttgtgcattcgattgcg

SEQ ID NO: 61 - DNA, 22 bp, synthetic construct
  source 1..22: mol_type = other DNA
gaagtctaccaaagctcacgcg

SEQ ID NO: 62 - DNA, 22 bp, synthetic construct
  source 1..22: mol_type = other DNA
ggaactccttctccagctctag

SEQ ID NO: 63 - DNA, 21 bp, synthetic construct
  source 1..21: mol_type = other DNA
aggaagctcgcggaccagtac

SEQ ID NO: 64 - DNA, 22 bp, synthetic construct
  source 1..22: mol_type = other DNA
ggtggtccttcttgtgctgcac

SEQ ID NO: 65 - DNA, 22 bp, synthetic construct
  source 1..22: mol_type = other DNA
agctagaggtgaagatccgcga

SEQ ID NO: 66 - DNA, 22 bp, synthetic construct
  source 1..22: mol_type = other DNA
gcaggacaatcctggagttctc

SEQ ID NO: 67 - DNA, 23 bp, synthetic construct
  source 1..23: mol_type = other DNA
gataatgggagcaaccaagtggc

SEQ ID NO: 68 - DNA, 22 bp, synthetic construct
  source 1..22: mol_type = other DNA
cagtatgtgccagcaggaagac

SEQ ID NO: 69 - DNA, 22 bp, synthetic construct
  source 1..22: mol_type = other DNA
gcttcagcaaggaggaggtcat

SEQ ID NO: 70 - DNA, 22 bp, synthetic construct
  source 1..22: mol_type = other DNA
tctggagttggcacttctcgct

SEQ ID NO: 71 - DNA, 20 bp, synthetic construct
  source 1..20: mol_type = other DNA
gagatggggaagatgctggg

SEQ ID NO: 72 - DNA, 20 bp, synthetic construct
  source 1..20: mol_type = other DNA
ggaggacacagtcagggaga

SEQ ID NO: 73 - DNA, 25 bp, synthetic construct
  source 1..25: mol_type = other DNA
tccttttcatgcaacacccaacact

SEQ ID NO: 74 - DNA, 25 bp, synthetic construct
  source 1..25: mol_type = other DNA
tccaaatgctggtaacactgtggtc

SEQ ID NO: 75 - DNA, 20 bp, synthetic construct
  source 1..20: mol_type = other DNA
gcagtgtgccaaaccagatg

SEQ ID NO: 76 - DNA, 20 bp, synthetic construct
  source 1..20: mol_type = other DNA
gggtttgccggggtacaata

SEQ ID NO: 77 - DNA, 20 bp, synthetic construct
  source 1..20: mol_type = other DNA
attctgcaggggccgagcct

SEQ ID NO: 78 - DNA, 20 bp, synthetic construct
  source 1..20: mol_type = other DNA
atcacacggagccgcttggc

SEQ ID NO: 79 - DNA, 20 bp, synthetic construct
  source 1..20: mol_type = other DNA
gctcatggctgaagatgagg

SEQ ID NO: 80 - DNA, 19 bp, synthetic construct
  source 1..19: mol_type = other DNA
caggcgcttgtccttcttc

SEQ ID NO: 81 - DNA, 24 bp, synthetic construct
  source 1..24: mol_type = other DNA
gggaacatagagaaagaggtctca

SEQ ID NO: 82 - DNA, 24 bp, synthetic construct
  source 1..24: mol_type = other DNA
aattaatctcagcttccccctaac

SEQ ID NO: 83 - DNA, 24 bp, synthetic construct
  source 1..24: mol_type = other DNA
ttgctcctagcctatctttcctta

SEQ ID NO: 84 - DNA, 24 bp, synthetic construct
  source 1..24: mol_type = other DNA
ctttagaattcctggacccttctc

SEQ ID NO: 85 - DNA, 24 bp, synthetic construct
  source 1..24: mol_type = other DNA
acgtttctgcaaagctgtctagtt

SEQ ID NO: 86 - RNA, 24 nt, synthetic construct
  source 1..24: mol_type = other RNA
ggcttcaaaccattcagtaacttc

(19) United States
(12) Patent Application Publication
(10) Pub. No.: US 2025/0235486 A1
(43) Pub. Date: Jul. 24, 2025

(54) Method of Producing Insulin-Producing Cells

(71) Applicant: Monash University, Clayton, Victoria (AU)

(72) Inventors: Assam El-Osta, Clayton, Victoria (AU); Keith Al-Hasani, Clayton, Victoria (AU); Ishant Khurana, Clayton, Victoria (AU); Harikrishnan Kaipananickal, Clayton, Victoria (AU); Scott Stuart Maxwell, Clayton, Victoria (AU); Jun Okabe, Clayton, Victoria (AU); Safiya Naina Marikar, Clayton, Victoria (AU)

(21) Appl. No.: 18/861,161

(22) PCT Filed: Apr. 28, 2023
(86) PCT No.: PCT/AU2023/050347

§ 371 Date: Oct. 28, 2024

(30) Foreign Application Priority Data
Apr. 29, 2022 (AU) 2022901132

(51) Int. Cl. A61K 35/39; A61P 5/50; C12N 5/071

(52) U.S. Cl. CPC A61K 35/39; A61P 5/50; C12N 5/0676; C12N 2501/065; C12N 2506/22

(57) ABSTRACT

The present invention relates to methods and uses for producing an insulin-producing cell from a pancreatic exocrine cell comprising contacting the pancreatic exocrine cell with an inhibitor of EZH2. The present invention also relates to methods and uses for preventing or treating a disease involving dysfunctional insulin production.

### CROSS-REFERENCE TO RELATED APPLICATIONS

This application claims priority to Australian provisional application no. 2022901132, filed on 29 Apr. 2022, the entire disclosure of which is hereby incorporated by reference.

### FIELD OF THE INVENTION

The present invention relates to methods and uses of producing insulin-producing cells. The present invention also relates to methods and uses for preventing or treating a disease involving dysfunctional insulin production.

### BACKGROUND OF THE INVENTION

Beta-cells (β-cells) are a type of endocrine cell found in pancreatic islets that synthetize, store, and release insulin. Beta-cells typically make up about 50-70% of the cells in human islets. Type 1 diabetes (T1 D) is an autoimmune disease that selectively destroys the insulin-producing β-cells in the pancreas. While symptoms usually do not appear before 80% of the β-cell mass has been destroyed, absolute destruction of these cells leads to the dependence on exogenous insulin administration for survival. Other types of diabetes such as type 2 diabetes (T2D) can also involve lack of insulin during disease progression.

Current therapies for T1 D diabetes focus on replacing the damaged β-cell mass in diabetic patients, involving either whole pancreas or islet transplantation. Although proven to be of clinical utility, these therapies face a real shortage of organ donors together with the associated side-effects of immunosuppressive drugs. Consequently, current research has focussed on β-cell replacement and this is an unmet need for diabetic patients. Studies have investigated the generation of β-cells from human pluripotent stem cells (hPSCs) such as human embryonic stem cells (hESCs), given their ability to proliferate infinitively and differentiate into many cell types including β-cells. However, human embryonic stem cells are not an ideal cell source due to practical and ethical barriers around their use.

Accordingly, there is a need for new or improved therapies that can be used for the treatment or prevention of a disease involving dysfunctional insulin production, for example diabetes and in particular T1 D.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method of producing an insulin-producing cell from a pancreatic exocrine cell, the method comprising:
contacting a pancreatic exocrine cell, or a cell population comprising a pancreatic exocrine cell, with an inhibitor of EZH2 for a sufficient time and under conditions to allow generation of an insulin-producing cell from the pancreatic exocrine cell,
thereby producing an insulin-producing cell, or a cell population comprising an insulin-producing cell.

In any aspect of the invention, the insulin-producing cell may be characterised by increased expression of one or more pancreatic progenitor markers, relative to a pancreatic exocrine cell that has not been contacted with the inhibitor of EZH2. In preferred embodiments, the one or more pancreatic progenitor markers are selected from Pdx1, Ngn3, Sox9 and Sox11.

In any aspect of the invention, the insulin-producing cell may be characterised by increased expression of one or more β-cell markers, relative to a pancreatic exocrine cell that has not been contacted with the inhibitor of EZH2. In preferred embodiments, the one or more β-cell markers are selected from Nkx6.1, MafA, Ins hnRNA and Ins.

In any aspect of the invention, the insulin-producing cell may be characterised by increased expression of one or more proliferation markers, relative to a pancreatic exocrine cell that has not been contacted with the inhibitor of EZH2. In preferred embodiments, the proliferation marker is Ki67.

In some embodiments, the insulin-producing cell is characterised by increased expression of one, 2, 3, 4, 5, 6, 7 or all 8 of Pdx1, Ngn3, Sox9, Sox11, Nkx6.1, MafA, Ins, Ins hnRNA and Ki67, relative to a pancreatic exocrine cell that has not been contacted with the inhibitor of EZH2.

In any aspect of the invention, the insulin-producing cell may be characterised by no change in expression of one or more of the following: one or more ductal markers; one or more acinar markers; and one or more glucose-sensor markers. In some embodiments, the ductal marker is Ck19. In some embodiments, the acinar marker is Amy2A. In some embodiments, the glucose-sensor marker is Txnip.

Preferably, the insulin-producing cell may be capable of secreting insulin upon stimulation with glucose. In some embodiments, the insulin-producing cell is capable of repeatedly performing glucose stimulated insulin secretion (GSIS).

In any aspect of the invention, the insulin-producing cell may be a β-cell or a β-cell-like cell.

In any aspect of the invention, the pancreatic exocrine cell may be an acinar cell or a ductal cell. In some embodiments, the pancreatic exocrine cell is a ductal cell. In one embodiment, the ductal cell is a ductal epithelial cell (eg a pancreatic ductal epithelial cell).

In any aspect of the invention, the cell population comprising a pancreatic exocrine cell may comprise or consist of one or both of acinar cells and ductal cells (eg ductal epithelial cells). In some embodiments, the cell population comprises or consists of ductal cells. In some embodiments, the cell population does not comprise embryonic pluripotent stem cells. In one embodiment, the cell population does not contain any induced pluripotent stem cells (iPSCs), or does not contain any cells derived from iPSCs.

In any aspect of the invention, the pancreatic exocrine cell, or the cell population comprising the pancreatic exocrine cell, may be obtained from pancreatic tissue. In some embodiments, the pancreatic tissue comprises or consists of pancreatic exocrine cells. In some embodiments, the pancreatic tissue comprises or consists of mature terminally differentiated pancreatic cells. In some embodiments, the pancreatic tissue does not comprise embryonic pluripotent stem cells.

In any aspect of the invention, the inhibitor of EZH2 may directly inhibit the enzymatic activity of EZH2. In some embodiments, the inhibitor of EZH2 occupies the site for co-substrate S-adenosylmethionine (SAM) in the binding pocket of EZH2. The inhibitor of EZH2 may be any EZH2 inhibitor as described herein.

In some embodiments, the inhibitor of EZH2 is a compound having the following structure:

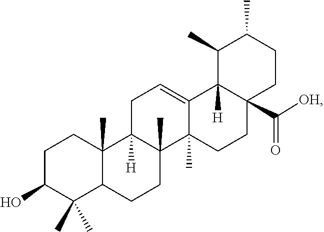

or a stereoisomer, a tautomer, a hydrate, a solvate, or a salt thereof, in particular a pharmaceutically acceptable salt, or a mixture of same.

In some embodiments, the inhibitor of EZH2 is a compound of formula (I):

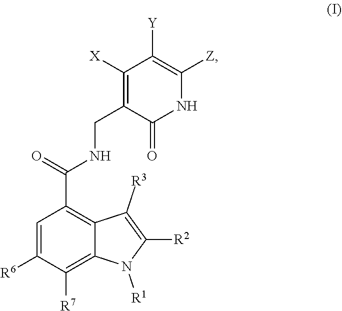

wherein
X and Z are selected independently from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halo, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, NR^{a}R^{b}, NR^{a}C(O)R^{b}, NR^{a}C(O)NR^{a}R^{b}, —NR^{a}C(O)OR^{a}, —NR^{a}SO₂R^{b}, NR^{a}SO₂NR^{a}R^{b}, NR^{a}NR^{a}R^{b}, NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};
Y is H or halo;
R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, —CONR^{a}NR^{a}R^{b};
R² is hydrogen, (C₁-C₈)alkyl, trifluoromethyl, alkoxy, or halo, in which said (C₁-C₈)alkyl maybe substituted with one to two groups selected from: amino, and (C₁-C₃)alkylamino;
R⁷ is hydrogen, (C₁-C₃)alkyl, or alkoxy; R³ is hydrogen, (C₁-C₈)alkyl, cyano, trifluoromethyl, —NR^{a}R^{b}, or halo;
R⁶ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-Cg)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, NR^{a}C(O)NR^{a}R^{b}, NR^{a}C(O)OR^{a}, NR^{a}SO₂R^{b}, NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, NR^{a}NR^{a}C(O)OR^{a}, —OR^{a}, —OC(O)R^{a}, —OC(O)NR^{a}R^{b};
   wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, NR^{a}C(O)R^{b}, —NR^{a}C(O)NR^{a}R^{b}, —NR^{a}C(O)OR^{a}, NR^{a}SO₂R^{b}, NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, NR^{a}R^{b}, NR^{a}C(O)R^{b}, NR^{a}C(O)NR^{a}R^{b}, NR^{a}C(O)OR^{a}, —NR^{a}SO₂R^{b}, NR^{a}SO₂NR^{a}R^{b}, —OR^{a}, —OC(O)R^{a}, and —OC(O)NR^{a}R^{b};
each R^{a} is independently (C₁-C₄)alkylamino, —NR^{a}SO₂R^{b}, —SOR^{a}, —SO₂R^{a}, —NR^{a}C(O)OR^{a}, —NR^{a}R^{b}, or —CO₂R^{a}; R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl)((C₁-C₄)alkyl);
or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁—C₄)alkoxy(C₁-C₄)alkyl, wherein said ring is optionally fused to a (C₃-C₈)cycloalkyl, heterocycloalkyl, aryl, or heteroaryl ring;
or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;
or a stereoisomer, a tautomer, a hydrate, a solvate, or a salt thereof, in particular a pharmaceutically acceptable salt, or a mixture of same.

In some embodiments, the compound of formula (I) is a compound having the following structure:

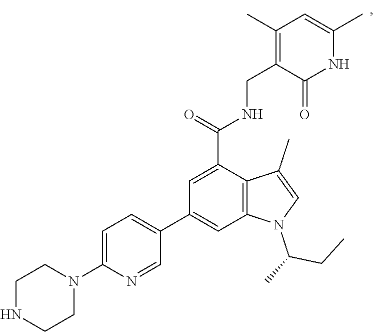 [SMILES: CC[C@H](C)n1cc(C)c2c(C(=O)NCc3c(C)cc(C)[nH]c3=O)cc(-c3ccc(N4CCNCC4)nc3)cc21]

or a stereoisomer, a tautomer, a hydrate, a solvate, or a salt thereof, in particular a pharmaceutically acceptable salt, or a mixture of same.

In some embodiments, the inhibitor of EZH2 is a compound having the following structure:

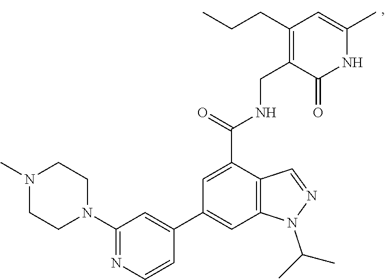

or a stereoisomer, a tautomer, a hydrate, a solvate, or a salt thereof, in particular a pharmaceutically acceptable salt, or a mixture of same.

In some embodiments, the inhibitor of EZH2 is a compound having the following structure:

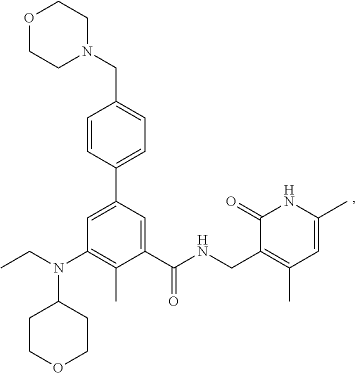 [SMILES: CCN(c1cc(-c2ccc(CN3CCOCC3)cc2)cc(C(=O)NCc2c(C)cc(C)[nH]c2=O)c1C)C1CCOCC1]

or a stereoisomer, a tautomer, a hydrate, a solvate, or a salt thereof, in particular a pharmaceutically acceptable salt, or a mixture of same.

In some embodiments, the inhibitor of EZH2 is a compound having the following structure:

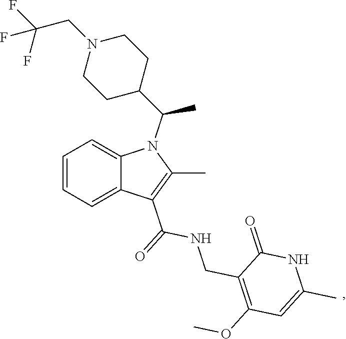 [SMILES: COc1cc(C)[nH]c(=O)c1CNC(=O)c1c(C)n([C@H](C)C2CCN(CC(F)(F)F)CC2)c2ccccc12]

or a stereoisomer, a tautomer, a hydrate, a solvate, or a salt thereof, in particular a pharmaceutically acceptable salt, or a mixture of same.

In some embodiments, the inhibitor of EZH2 is a compound having the following structure:

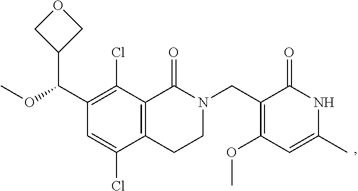

or a stereoisomer, a tautomer, a hydrate, a solvate, or a salt thereof, in particular a pharmaceutically acceptable salt, or a mixture of same.

In some embodiments, the inhibitor of EZH2 is a compound having the following structure:

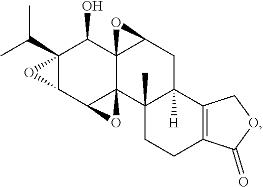

or a stereoisomer, a tautomer, a hydrate, a solvate, or a salt thereof, in particular a pharmaceutically acceptable salt, or a mixture of same.

In some embodiments, the inhibitor of EZH2 is a compound having the following structure:

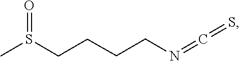

or a stereoisomer, a tautomer, a hydrate, a solvate, or a salt thereof, in particular a pharmaceutically acceptable salt, or a mixture of same.

In preferred embodiments, the inhibitor of EZH2 is selected from one of the following:

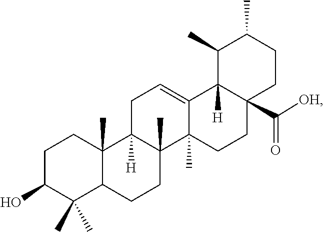

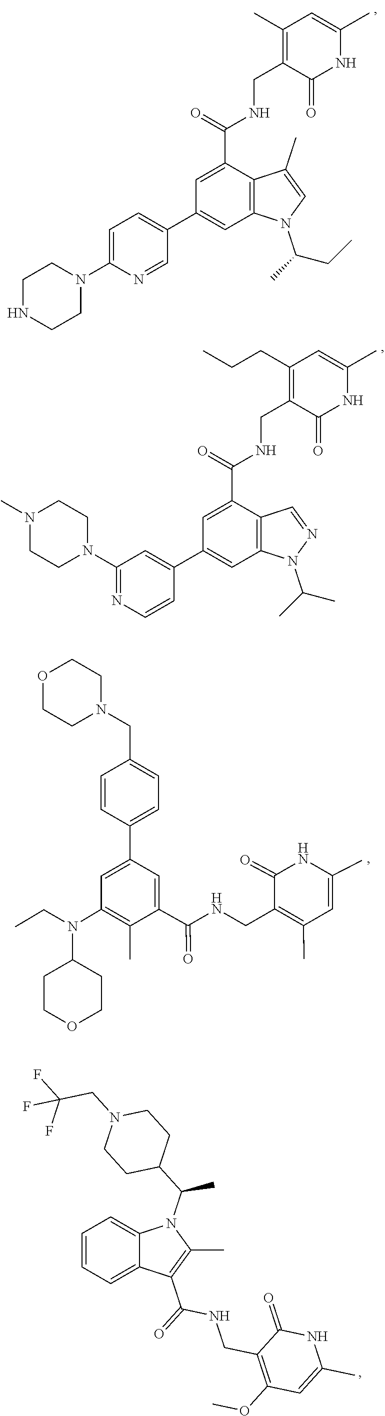

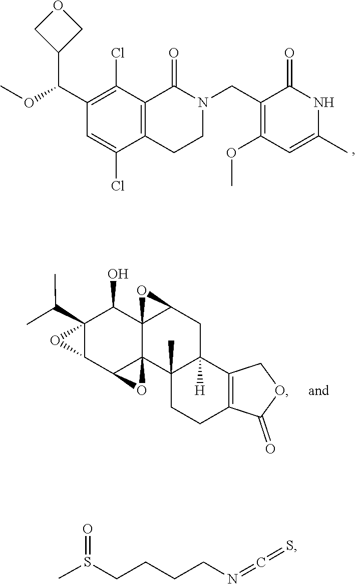

or a stereoisomer, a tautomer, a hydrate, a solvate, or a salt thereof, in particular a pharmaceutically acceptable salt, or a mixture of same.

The method may further comprise a step of administering the insulin-producing cell, or the cell population comprising the insulin-producing cell, to an individual. In some embodiments, the pancreatic exocrine cell from which the insulin-producing cell is generated is obtained from the individual. In some embodiments, the individual has diabetes mellitus or is pre-diabetic. In some embodiments, the diabetes mellitus is selected from type 1 diabetes, type 2 diabetes and gestational diabetes.

In another aspect, the present invention provides an insulin-producing cell, or a cell population comprising an insulin-producing cell, produced by a method as described herein.

In another aspect, the present invention provides the use of an inhibitor of EZH2 for producing an insulin-producing cell from a pancreatic exocrine cell.

In another aspect, the present invention provides the use of an inhibitor of EZH2 in the manufacture of a composition for producing an insulin-producing cell from a pancreatic exocrine cell.

In another aspect, the present invention provides an inhibitor of EZH2 for use in producing an insulin-producing cell from a pancreatic exocrine cell.

In another aspect, the present invention provides a method for preventing or treating a disease involving dysfunctional insulin production, the method comprising:
administering an insulin-producing cell, or a cell population comprising an insulin-producing cell, as described herein or produced by a method as described herein, to an individual in need thereof,
thereby preventing or treating the disease.

In another aspect, the present invention provides a method for preventing or treating a disease involving dysfunctional insulin production in an individual in need thereof, the method comprising:
obtaining a pancreatic exocrine cell, or a cell population comprising a pancreatic exocrine cell, from pancreatic tissue of an individual,
contacting the pancreatic exocrine cell, or the cell population comprising a pancreatic exocrine cell, with an inhibitor of EZH2 for a sufficient time and under conditions to allow generation of an insulin-producing cell from the pancreatic exocrine cell, thereby producing an insulin-producing cell, or a cell population comprising an insulin-producing cell, and
administering the insulin-producing cell, or the cell population comprising an insulin-producing cell to the individual,
thereby preventing or treating the disease.

In another aspect, the present invention provides the use of an insulin-producing cell, or a cell population comprising an insulin-producing cell, as described herein or produced by a method as described herein, in the manufacture of a composition for preventing or treating a disease involving dysfunctional insulin production in an individual.

In another aspect, the present invention provides an insulin-producing cell, or a cell population comprising an insulin-producing cell, as described herein or produced by a method as described herein, for use in preventing or treating a disease involving dysfunctional insulin production in an individual.

The disease involving dysfunctional insulin production may be selected from diabetes mellitus and pre-diabetes. In some embodiments, the diabetes mellitus is selected from type 1 diabetes, type 2 diabetes and gestational diabetes.

The pancreatic exocrine cell from which the insulin-producing cell is generated may be from pancreatic tissue obtained from the individual.

The insulin-producing cell, or the cell population comprising the insulin-producing cell, may be delivered to the pancreas of the individual.

In another aspect, the present invention provides a method for preventing or treating a disease involving dysfunctional insulin production, the method comprising:
administering an inhibitor of EZH2 to an individual in need thereof such that the inhibitor of EZH2 contacts a pancreatic exocrine cell of the individual for a sufficient time and under conditions to allow generation of an insulin-producing cell from the pancreatic exocrine cell,
thereby preventing or treating the disease in the individual.

In another aspect, the present invention provides the use of an inhibitor of EZH2 in the manufacture of a composition for preventing or treating a disease involving dysfunctional insulin production in an individual, wherein the inhibitor of EZH2 contacts a pancreatic exocrine cell of the individual for a sufficient time and under conditions to allow generation of an insulin-producing cell from the pancreatic exocrine cell.

In another aspect, the present invention provides an inhibitor of EZH2 for use in preventing or treating a disease involving dysfunctional insulin production in an individual, wherein the inhibitor of EZH2 contacts a pancreatic exocrine cell of the individual for a sufficient time and under conditions to allow generation of an insulin-producing cell from the pancreatic exocrine cell.

In any aspect or embodiment, the disease involving dysfunctional insulin production may be selected from diabetes mellitus and pre-diabetes. In some embodiments, the diabetes mellitus is selected from type 1 diabetes, type 2 diabetes and gestational diabetes.

In any aspect or embodiment, the inhibitor of EZH2 may be any EZH2 inhibitor as described herein.

In any aspect or embodiment, the inhibitor of EZH2 may be orally administered to the individual.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. (a) Representative immunohistochemical insulin and glucagon staining in a non-diabetic (ND) donor and type 1 diabetes (T1 D) donor. Insulin and glucagon expression are indicated by the darker grey staining in human islets. Note the complete absence of insulin in the T1D donor; (b) Schematic organisation of the exocrine and endocrine compartments in the human pancreas featuring the ductal epithelial cells which are hypothesized to contain progenitor cells capable of regeneration upon exposure to EZH2 inhibitors (EZH2i).

FIG. 2. Distinguishable expression of embryonic and β-cell mRNA indices from naïve pancreatic exocrine cells isolated from donors. (a) Expression analyses in naïve exocrine cells showing the comparative abundance of Ins, Pdx1, Ngn3, Sox9, Sox11, Txnip, Ck19 and Amy2A mRNA relative to 18s assessed by qRT-PCR. Relative expression of mRNA isolated from non-diabetic (nd) and type 1 diabetic (T1D) donors. (b) Correlation of mRNA expression of Ins, Pdx1, Ngn3, Sox9, Sox11, Txnip, Ck19 and Amy2A displayed as a function of fold change. Expression analyses of mRNA isolated from non-diabetic (nd) and type 1 diabetic (T1D) donors. Non-diabetic donor 1 (nd1) depicted as hashed bars, nd donor 2 (nd2) depicted as unfilled bars, T1 D donor depicted as filled bars.

FIG. 3. Pharmacological inhibition of EZH2 activity by GSK126 diminishes H3K27me3 and not H3K27ac protein content. (a) Dose dependent increase of GSK126 attenuates H3K27me3 in human pancreatic ductal epithelial cells. Bar graph represents signal ratio values of H3K27me3 protein adjusted against histone H3 by Li—COR Odyssey quantitation. Vehicle control is DMSO. (b) Dose dependent increase of GSK126 does not attenuate H3K27ac in human pancreatic ductal epithelial cells.

FIG. 4. GSK126 restores expression of islet indices in non-diabetic and type 1 diabetic pancreatic exocrine cells. Correlation of mRNA abundance of Ngn3, Sox9, Sox11, Ins, Pdx1, Ck19, Amy2A, Txnip, Mafa, Nkx6.1, Igf2 and Igf2AS displayed as fold change by qRT-PCR. Significant changes in mRNA abundance were not detected for the ductal marker (Ck19), acinar marker (Amy2A) and the glucose-sensor (Txnip). Student's t-test was performed on Control vs GSK126 (*P<0.05, **P<0.01, ***P<0.001, ***P<0.0001 error bars are SEM, n=3).

FIG. 5. GSK126 influences the refractory H3K27me3 content on the insulin chromatin domain in human non-diabetic exocrine cells. (a) Insulin domain was assessed using amplimers that were specifically designed to distinguish promoter regions (R) of Ins and Igf2AS genes. (b) Quantitative PCR analysis of DNA in chromatin immunoprecipitated (ChIP) with anti-H3K27me3 antibody. Vehicle control was DMSO. (c) DNA was assessed using amplimers that specifically recognise the promoter regions (R) of Ngn3 and Pdx1 genes. (d) Quantitative PCR analysis of DNA in ChIP with anti-H3K27me3 antibody. Vehicle control was DMSO. Control samples depicted as filled bars, GSK126 samples depicted as unfilled bars. Data represented as the mean Input signal against specific H3K27me3 abundance. Student's t-test was performed on Control vs GSK126 (*P<0.05, **P<0.01 error bars are SEM, n=2).

FIG. 6. Schematic representation of pancreatic progenitor differentiation and the refractory influence of H3K27me3 content on the insulin chromatin domain in human exocrine cells. (a) Organisation of the human endocrine and exocrine pancreas showing the main pancreatic ductal tree connecting the acinar bundle. (b) The inability to influence transcriptional expression in the exocrine and endocrine pancreas is in accordance with default transcriptional suppression mediated by EZH2 dependent H3K27me3. Conversion of default repression state is influenced by pharmacological inhibition of EZH2 by GSK126 to prime β-cell lineage regeneration and restore insulin expression. (c) Proposed model of the Ins chromatin domain in pancreatic cells. Default transcriptional suppression is characterised with H3K27me3 rich regions can influence chromatin conformation, and suppress the expression of Ins, Igf2AS and Ins-Igf2 genes postulated by long distance chromatinised-looping.

FIG. 7. Ursolic acid and GSK126 influence the expression of β-cell and progenitor indices in human pancreatic ductal epithelial cells outside the islets of Langerhans. Correlation of mRNA abundance of Insulin, Pdx1 and Ngn3, displayed as fold change by qRT-PCR.

FIG. 8. Effect of GSK126, CPI-1205 (CPI), ursolic acid (UA), GSK343 and EPZ-6438 (EPZ) on mRNA expression of Insulin (depicted as hashed bars), Pdx1 (depicted as unfilled bars) and Ngn3 (depicted as filled bars) in acinar and duct (AD) cells from a non-diabetic donor. Drug treatment for 48 hours: GSK126 10 μM, CPI 5 μM, UA 5 μM, GSK343 5 μM and EPZ 5 μM.

FIG. 9. Effect of GSK126 and ursolic acid (UA) on mRNA expression of Insulin (depicted as hashed bars), Pdx1 (depicted as unfilled bars) and Ngn3 (not depicted; no amplification) in acinar and duct (AD) cells from a marmoset donor. Drug treatment for 48 hours: GSK126 10 μM and UA 5 μM.

FIG. 10. Key markers of endocrine development, epithelial-to-mesenchymal transition, pancreatic ducts, and islets are altered in human pancreatic ductal cells following stimulation with EZH2 inhibitors. From left to right the data represents the control, GSK126, EPZ6438 and Triptolide respectively. Variation in mRNA transcripts of (A) Neurogenin3 [NGN3], (B) Pancreatic duodenal homeobox factor 1 [PDX1], (C) SRY-box transcription factor 9 [SOX9], (D) Alpha amylase [AMY2A], (E) Cytokeratin 19 [CK19], (F) Insulin heterogenous nuclear RNA [INS hnRNA], (G) Insulin [INS] and (H) V-maf musculoaponeurotic fibrosarcoma oncogene homolog A [MAFA], following 2- and 7-day stimulation with GSK-126 at 10 μM, EPZ6438 at 1 μM, and Triptolide at 20 nM. Data are displayed as mean of fold change±S.E.M. of 3 replicates, calculated by normalizing drug values to DMSO (vehicle treated) controls. Statistically significant change in expression was determined using Student's t-test to compare control values to each drug, *P<0.05, **P<0.01, ***P<0.001, ****P<0.0001.

FIG. 11. Stimulation with EZH2 inhibitors reduces H3K27me3 content in human pancreatic ductal epithelial cells. (A) Histone proteins were extracted from pancreatic ductal cells stimulated with EZH2 inhibitors and control cells using 5 M of sulfuric acid. Acid precipitated (ppt) histone proteins were separated on Nu-Page gel followed by immunoblotting to quantify the total H3 and H2K27me3 levels using Li—CoR Odyssey. (B) Representative western blots of H3K27me3, and H3K27ac relative to total H3 following 2-day and 7-day stimulation with GSK126 at 10 μM, EPZ6438 at 1 μM, Triptolide at 20 nM compared with vehicle control DMSO. (C) Quantitative analysis of H3K27ac, and (D) H3K27me3 relative to total H3 following 2-day and 7-day stimulation with GSK126 at 10 μM, EPZ6438 at 1 μM, Triptolide at 20 nM compared with vehicle control DMSO. Data are displayed as mean signal ratio of H3K27me3 to total H3±SEM of 3 replicates with representative blots above. Each dot plot represents signal ratio of H3K27ac from one independent replicate. Each triangle plot represents signal ratio of H3K27me3 from one independent replicate. Statistically significant differences were determined using Student's t-tests against control. *P<0.05, **P<0.01.

FIG. 12. Reduction of H3K27me3 content associated with the chromatin of DNA in the INS-IGF2, NGN3, and PDX1 promoter regions following inhibition of EZH2. (a) H3K27me3 content was assessed by using amplifiers (black bars against DNA regions corresponding to the promoters of INS, NGN3 and PDX1). Quantitative PCR analysis of H3K27me3 associated DNA using ChIP following (b) 2-day and (c) 7-day stimulation of human pancreatic ductal epithelial cells compared to vehicle control. Data are displayed as the mean input signal against H3K27me3 abundance±S.E.M of 3 replicates. Each triangle plot represents one technical replicate. Statistically significant differences were determined using Student's t-tests against control. *P<0.05, **P<0.01, ***P<0.001. Quantitative PCR analysis of H3K9/14ac associated DNA using ChIP following (D) 2-day and (E) 7-day stimulation of human pancreatic ductal epithelial cells compared to vehicle control. Data are displayed as the mean input signal against H3K9/14ac abundance±S.E.M of 3 replicates. Each dot plot represents one technical replicate. Statistically significant differences were determined using Student's t-tests against control. Graphical data from left to right corresponds to the control, GSK126, EPZ6438 and triptolide respectively.

FIG. 13. EZH2 inhibitors influence glucose sensitive insulin secretion in human pancreatic ductal cells. (a) Gene expression of chromatin modulators EZH2, P300, KAT2, and BRG1 are unchanged in non-inhibitor treated cells following exposure to high glucose (HG) compared to non-exposed (LG) cells. Data are presented as mean of fold change±S.E.M. of 2 replicates, calculated by normalizing high glucose values to low glucose (unexposed) controls. Statistically significant change in expression was determined using Student's t-test. (b) Representative western blots and quantitative analysis of EZH2 and H3K27me3 following exposure to high glucose (HG) compared to low glucose (LG). Data are displayed as mean signal ratio of EZH2 to β-actin or H3K27me3 to total H3±SEM of 2 replicates with representative blots above. Each dot plot represents signal ratio of one independent replicate. Statistically significant differences were determined using Student's t-tests against control. (c) 2- and 7-day protocols for assessment of glucose stimulated insulin secretion from EZH2 inhibitor (EZH2i) treated HPDE cells. Both protocols were initiated with seeding of cells to establish cultures. 2-day EZH2i stimulation was performed in CMRL to resolve background insulin, whilst for 7-day stimulations, the initial EZH2i doses were delivered in normal growth media, followed by switching to CMRL on day 6. On the final day of the protocol, cells were incubated for one hour in low glucose followed by one hour in high glucose. The supernatant was collected for quantification of insulin secretion in ELISAs. ELISA quantified (d) 2- and (e) 7-day secretion of insulin from HPDE cells following one hour of incubation in low (2.8 mM—left/circle) and high (28 mM—right/triangle) concentrations of glucose. Insulin concentrations were normalized to control 2.8 mM concentrations to calculate fold change. Data are presented as mean of fold change±S.E.M. of 3 replicates. Filled circles represent one technical replicate of 2.8 mM glucose supernatant. Triangles represent one technical replicate of 28 mM glucose supernatant. Student's t-tests were used to assess if variation in insulin secretion was statistically significant, *P<0.05, **P<0.01, ****P<0.0001.

FIG. 14. Graph demonstrating a lack of change in the H3K9/14 acetylation (H3K9/14ac) demonstrating the specificity of the EZH2i in modulating the trimethylation signal of HPDE cells. Quantitative analysis of H3K9/14ac relative to total H3 following 2-day and 7-day stimulation with GSK126 at 10 μM, EPZ6438 at 1 μM, Triptolide at 20 nM compared with vehicle control DMSO. Data are displayed as mean signal ratio of H3K9/14ac to total H3±SEM of 3 replicates. Statistically significant differences were determined using Student's t-tests against control.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For the purposes of interpreting this specification, terms used in the singular will also include the plural and vice versa.

The present inventors have identified that functional insulin-producing cells can be produced by inhibiting EZH2 (enhancer of zeste homolog 2) in human pancreatic exocrine cells. Previous studies have shown that inhibition of EZH2 can allow differentiation and generation of β-cells from pluripotent stem cells. The present inventors have demonstrated for the first time the production of functional insulin-producing cells from mature terminally differentiated pancreatic exocrine cells, including functionally damaged mature terminally differentiated pancreatic exocrine cells, obtained from pancreatic tissue of non-diabetic and type-1 diabetic donors. As discussed herein and shown in the Examples, stimulation of pancreatic exocrine cells with an EZH2 inhibitor increased the expression of core β-cell determinants, including insulin transcription. The results described herein include the first example of restoring exocrine transcription of the insulin gene in exocrine cells derived from an insulin devoid T1 D donor. These findings supports the use of exocrine cells from the pancreas as a source for generating functional insulin-producing cells.

Advantageously, the methods and uses described herein do not rely on stem cells, and do not require specific culturing conditions to influence β-cell production from embryonic stem cells. Further, in contrast to the 3-4 months typically required to produce β-cells from embryonic stem cells, the methods and uses described herein can allow for rapid production of insulin-producing cells (e.g., within 1-4 days). Moreover, since the methods and uses described herein can allow for the production of insulin-producing cells from an individual's own pancreatic exocrine cells, immunosuppressive therapy may not be required.

### Insulin-Producing Cells

The present invention provides a method of producing an insulin-producing cell from a pancreatic exocrine cell, the method comprising: contacting a pancreatic exocrine cell, or a cell population comprising a pancreatic exocrine cell, with an inhibitor of EZH2 for a sufficient time and under conditions to allow generation of an insulin-producing cell from the pancreatic exocrine cell, thereby producing an insulin-producing cell, or a cell population comprising an insulin-producing cell. As described herein and as shown in the Examples, the methods and uses of the invention are capable of providing functional insulin-producing cells.

The present invention also provides the use of an inhibitor of EZH2 for producing an insulin-producing cell from a pancreatic exocrine cell. The invention also provides the use of an inhibitor of EZH2 in the manufacture of a composition for producing an insulin-producing cell from a pancreatic exocrine cell. The invention also provides an inhibitor of EZH2 for use in producing an insulin-producing cell from a pancreatic exocrine cell. Preferably, the inhibitor of EZH2 is contacted with a pancreatic exocrine cell for a sufficient time and under conditions to allow generation of an insulin-producing cell from the pancreatic exocrine cell.

The insulin-producing cell, or cell population comprising the insulin-producing cell, as produced by the method may be characterised by one or more of the following:
increased expression of one or more pancreatic progenitor markers, for example increased expression of any one or more of Pdx1, Ngn3, Sox9 and Sox11;
increased expression of one or more β-cell markers, for example increased expression of any one or more of Nkx6.1, MafA, Ins hnRNA and Ins;
increased expression of one or more proliferation genes, for example increased expression of Ki67;
no change in expression of one or more ductal markers, for example no change in expression of Ck19;
no change in expression of one or more acinar markers, for example no change in expression of Amy2A; and
no change in expression of one or more glucose-sensor markers, for example no change in expression of Txnip;
relative to expression in a pancreatic exocrine cell that has not been contacted with the inhibitor of EZH2.

In some embodiments, the insulin-producing cell, or cell population comprising the insulin-producing cell, as produced by the method is characterised by one or both of the following:
increased expression of any one or more of Pdx1, Ngn3, Sox9, Sox11, Nkx6.1, MafA, Ins, Ins hnRNA and Ki67; and
no increase in expression of any one or more of Ck19, Amy2A and Txnip;
relative to expression in a pancreatic exocrine cell that has not been contacted with the inhibitor of EZH2.

The insulin-producing cell is preferably capable of secreting insulin upon stimulation with glucose, that is, capable of glucose stimulated insulin secretion (GSIS). In some embodiments, the insulin-producing cell is capable of repeatedly performing GSIS. GSIS may be assessed by methods known in the art, for example by treating cells with low (e.g., 2.5 mM) or high (e.g., 25 mM) concentration of glucose and examining insulin release by ELISA (enzyme-linked immunosorbent assay).

The insulin-producing cell as produced by the method may be a β-cell or a β-cell-like cell. As used herein, “β-cell-like cell” is intended to encompass a cell that exhibits one or more characteristics of insulin-producing cells as described herein.

Typically, conditions suitable for generating an insulin-producing cell from a pancreatic exocrine cell include culturing the pancreatic exocrine cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. In some embodiments, the pancreatic exocrine cell may be contacted with the inhibitor of EZH2 for 1 to 30 days, preferably 1 to 14 days, more preferably 1 to 7 days, even more preferably 1 to 4 days. In some embodiments, the pancreatic exocrine cell may be contacted with the inhibitor of EZH2 for about 2 days, or about 7 days, or from about 2 days and about 7 days. In some embodiments, the pancreatic exocrine cell may be contacted with the inhibitor of EZH2 for 2 days, or 7 days, or from 2 days and 7 days. Advantageously, as shown in the Examples, the methods and uses of the invention allow for rapid generation of insulin-producing cells within 2 days after treatment with EZH2 inhibitors. Any medium suitable for culturing pancreatic cells may be used. Examples of suitable media include Keratinocyte SFM (serum-free medium) and Miami/CMRL medium as described in the Examples, and other suitable media in the art such as RPMI medium (also referred to as Roswell Park Memorial Institute (RPMI) 1640 medium). Advantageously, the methods and uses of the invention do not require specific culture conditions to influence generation of insulin-producing cells, such as those required for generation of β-cells from embryonic stem cells.

The present invention also provides an insulin-producing cell produced by a method as described herein.

One, or more, or all steps of the herein described methods may be performed in vitro.

The present invention also provides a cell population comprising an insulin-producing cell produced by a method as described herein. In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of cells are insulin-producing cells, and those cells are produced by a method as described herein.

The present invention also provides a composition comprising an insulin-producing cell, or a cell population comprising an insulin producing cell, produced by the method described herein.

### Exocrine Pancreatic Cells

Exocrine pancreatic cells are pancreatic cells that secrete digestive enzymes, water and bicarbonate into the small intestine to assist in digestion. The majority of the pancreas is made up of the exocrine portion (about 85% by mass), which includes acinar and duct tissue. As described herein and as shown in the Examples, the methods and uses of the invention are capable of providing functional insulin-producing cells from pancreatic exocrine cells.

In some embodiments, the pancreatic exocrine cell is an acinar cell or a ductal cell, preferably a ductal cell.

Preferably, the pancreatic exocrine cell is a mature (or adult) terminally differentiated pancreatic exocrine cell. Preferably, the pancreatic exocrine cell is not a pluripotent stem cell, for example an embryonic pluripotent stem cell.

In some embodiments, the cell population comprising a pancreatic exocrine cell comprises or consists of one or both of acinar cells and ductal cells. In this context, “consists of” will be understood to imply that the cell population only includes one or both of acinar cells and ductal cells, and no other types of pancreatic cells (e.g., endocrine or islet cells). In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells in the cell population are one or both of acinar cells and ductal cells. In some embodiments, the cell population does not comprise endocrine cells (or islet cells).

In preferred embodiments, the cell population comprises ductal cells. In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells in the cell population are ductal cells. In some embodiments, the cell population consists of ductal cells. In this context, “consists of” will be understood to imply that the cell population only includes ductal cells, and no other types of cells.

Preferably, the cell population comprises mature (or adult) terminally differentiated pancreatic cells. In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells in the cell population are mature terminally differentiated pancreatic cells. In some embodiments, the cell population consists of mature terminally differentiated pancreatic cells. In this context, “consists of” will be understood to imply that the cell population only includes mature terminally differentiated pancreatic cells, and no other types of cells (e.g., pluripotent stem cells such as embryonic pluripotent stem cells). Preferably, the cell population does not comprise pluripotent stem cells, for example embryonic pluripotent stem cells.

The mature terminally differentiated pancreatic cells may comprise or consist of pancreatic exocrine cells. In this context, “consists of” will be understood to imply that the mature terminally differentiated pancreatic cells only includes pancreatic exocrine cells, and no other types of cells (e.g., endocrine or islet cells). In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells in the cell population are mature terminally differentiated pancreatic exocrine cells.

The mature terminally differentiated pancreatic exocrine cells may be one or both of acinar cells and ductal cells. In some embodiments, the mature terminally differentiated pancreatic cells comprise or consist of ductal cells. In this context, “consists of” will be understood to imply that the mature terminally differentiated pancreatic cells only includes ductal cell, and no other types of cells. In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells in the cell population are mature terminally differentiated ductal cells.

The pancreatic exocrine cell, or the cell population comprising the pancreatic exocrine cell, may be obtained from any suitable source. In some embodiments, the pancreatic exocrine cell, or the cell population comprising the pancreatic exocrine cell, is obtained from pancreatic tissue.

The pancreatic tissue may be obtained from any suitable source. For example, the pancreatic tissue may be obtained from a biopsy, from surgery or from a cadaver. The pancreatic tissue may be human pancreatic tissue or non-human pancreatic tissue, for example non-human primate pancreatic tissue. Advantageously, as shown in the examples, treatment with EZH2 inhibitors increased expression of key β-cell determinants including insulin transcription in exocrine cells from human and marmoset pancreatic tissue samples.

The pancreatic tissue may comprise or consist of exocrine cells. In some embodiments, the pancreatic tissue comprises or consists of one or both of acinar cells and ductal cells. In this context, “consists of” will be understood to imply that the pancreatic tissue only includes one or both of acinar cells and ductal cells, and no other types of pancreatic cells (e.g., endocrine or islet cells). In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells making up the pancreatic tissue are one or both of acinar cells and ductal cells. In some embodiments, the pancreatic tissue does not comprise endocrine cells (or islet cells).

In preferred embodiments, the pancreatic tissue comprises ductal cells. In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells making up the pancreatic tissue are ductal cells. In some embodiments, the pancreatic tissue consists of ductal cells. In this context, “consists of” will be understood to imply that the pancreatic tissue only includes ductal cells, and no other types of cells.

Preferably, the pancreatic tissue comprises mature (or adult) terminally differentiated pancreatic cells. In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells making up the pancreatic tissue are mature terminally differentiated pancreatic cells. In some embodiments, the pancreatic tissue consists of mature terminally differentiated pancreatic cells. In this context, “consists of” will be understood to imply that the pancreatic tissue only includes mature terminally differentiated pancreatic cells, and no other types of cells (e.g., pluripotent stem cells such as embryonic pluripotent stem cells). Preferably, the pancreatic tissue does not comprise pluripotent stem cells, for example embryonic pluripotent stem cells.

The mature terminally differentiated pancreatic cells may comprise or consist of pancreatic exocrine cells. In this context, “consists of” will be understood to imply that the mature terminally differentiated pancreatic cells only includes pancreatic exocrine cells, and no other types of cells (e.g., endocrine or islet cells). In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells making up the pancreatic tissue are mature terminally differentiated pancreatic exocrine cells.

The mature terminally differentiated pancreatic exocrine cells may be one or both of acinar cells and ductal cells. In some embodiments, the mature terminally differentiated pancreatic cells comprise or consist of ductal cells. In this context, “consists of” will be understood to imply that the mature terminally differentiated pancreatic cells only includes ductal cell, and no other types of cells. In some embodiments, at least about 5%, about 10%, about 15%, about 20%, about 25%, about 30%, about 35%, about 40%, about 45%, about 50%, about 55%, about 60%, about 65%, about 70%, about 80%, about 85%, about 90%, about 91%, about 92%, about 93%, about 94%, about 95%, about 96%, about 97%, about 98%, about 99% or about 100% of the cells in the pancreatic tissue are mature terminally differentiated ductal cells.

### EZH2 and Inhibitors of EZH2

EZH2 (enhancer of zeste homolog 2) is a histone-lysine N-methyltransferase involved in transcriptional repression. EZH2 is the functional catalytic subunit of PRC2 (Polycomb Repressive Complex 2) and catalyses methylation of histone 3 at Lys 27 (H3K27me3) using cofactor S-adenosylmethionine (SAM).

As used herein, an “inhibitor of EZH2” or an “EZH2 inhibitor” is any molecule that inhibits the activity of EZH2, for example, completely or partially reduces one or more functions of EZH2 including those described herein. The molecule may inhibit the enzymatic activity of EZH2, for example by binding the active site, or competing with the enzyme substrate or co-effector or signalling mechanism. In some embodiments, the inhibitor of EZH2 occupies, including partially or completely, the site for co-substrate S-adenosylmethionine (SAM) in the binding pocket of EZH2. In some embodiments, the inhibitor of EZH2 competes with SAM for binding to EZH2.

The EZH2 inhibitor may be specific for EZH2 and only have some low level inhibitory activity against other enzymes. For example, the EZH2 inhibitor may have a Ki of greater than about 50 μM or 100 μM, preferably 1 mM against other enzymes as measured using an assay as described herein, or a Ki against other enzymes at least 10 times greater than the Ki against EZH2.

In one embodiment, the inhibitor of EZH2 has greater than 5, 10, 15, 20, 25, 30, 35, 40, 45, 50, 55, 60, 65, 70, 75, 80, 85, 90, 95, 100, 110, 120, 130, 140 or 150 fold selectivity for EZH2 over EZH1.

Preferably, the inhibitor of EZH2 is a molecule that limits the activity of EZH2 to 10% or less in comparison with control. Control is a solvent, in which the inhibitor is tested, used at the same quantity, however, without the inhibitor. The enzymatic activities of EZH2 may be determined by methylation of H3 (histone 3) by 5 member PRC2 complex (Flag-EZH2, EED, SUZ12, AEBP2, RbAp48) in the presence of [³H]-SAM (tritiated SAM, S-adenosyl-L-[methyl-³H]methionine), monitored by detection of the tritiated histone product (histone N⁶-[methyl-³H]-L-lysine) according to McCabe et al (Nature, 2012, 492(7427), 108-112), Verma et al (ACS Med. Chem. Lett. 2012, 3, 12, 1091-1096), Knutson et al (Proc Natl Acad Sci USA, 2013 May 7; 110(19):7922-7), Vaswani et al (J Med Chem, 2016, 59, 21, 9928-9941), Kung et al (J Med Chem, 2018, February 8; 61(3):650-665), WO2011/140324 (US20130053397A1), WO2011/140325 (US20130053383A1), WO2012/142504 (US20120264734A1), WO2013/120104 (US20150011546A1), WO2015/193765 (US20150361067A1), or other method described herein.

The inhibitor of EZH2 may exhibit a Ki value of less than 1 mM, preferably less than 100 μM, more preferably less than 10 μM, as determined by an assay as described herein, for example an assay of methylation activity. In some embodiments, the EZH2 inhibitor exhibits a Kᵢ value of equal to or less than about 1 mM, equal to or less than about 900 μM, equal to or less than about 800 μM, equal to or less than about 700 μM, equal to or less than about 600 μM, equal to or less than about 500 μM, equal to or less than about 400 μM, equal to or less than about 300 μM, equal to or less than about 200 μM, equal to or less than about 100 μM, equal to or less than about 90 μM, equal to or less than about 80 μM, equal to or less than about 70 μM, equal to or less than about 60 μM, equal to or less than about 50 μM, equal to or less than about 40 μM, equal to or less than about 30 μM, equal to or less than about 20 μM, equal to or less than about 10 μM, equal to or less than about 1 μM, equal to or less than about 900 nM, equal to or less than about 800 nM, equal to or less than about 700 nM, equal to or less than about 600 nM, equal to or less than about 500 nM, equal to or less than about 400 nM, equal to or less than about 300 nM equal to or less than about 250 nM, equal to or less than about 200 nM, equal to or less than about 150 nM, equal to or less than about 100 nM, equal to or less than about 50 nM or equal to or less than about 10 nM, or any equivalent unit of measure (e.g., mol/L), as determined by an assay of methylation activity. The EZH2 inhibitor may exhibit a Kᵢ value of equal to or less than any value defined herein. By way of example, the EZH2 inhibitor may be EPZ-6438, which exhibits a Kᵢ of about 2.5 nM as determined by an assay of methylation activity as described in Knutson et al (Proc Nat/Acad Sci USA, 2013 May 7; 110(19):7922-7).

Additionally, or alternatively, the inhibitor of EZH2 may exhibit an IC₅₀ value of less than about 1 mM, preferably less than about 100 μM, more preferably less than about 10 μM, as determined by an assay as described herein, for example an assay of methylation activity. In some embodiments, the EZH2 inhibitor exhibits an IC₅₀ value of equal to or less than about 1 mM, equal to or less than about 900 μM, equal to or less than about 800 μM, equal to or less than about 700 μM, equal to or less than about 600 μM, equal to or less than about 500 μM, equal to or less than about 400 μM, equal to or less than about 300 μM, equal to or less than about 200 μM, equal to or less than about 100 μM, equal to or less than about 90 μM, equal to or less than about 80 μM, equal to or less than about 70 μM, equal to or less than about 60 μM, equal to or less than about 50 μM, equal to or less than about 40 μM, equal to or less than about 30 μM, equal to or less than about 20 μM, equal to or less than about 10 μM, equal to or less than about 1 μM, equal to or less than about 900 nM, equal to or less than about 800 nM, equal to or less than about 700 nM, equal to or less than about 600 nM, equal to or less than about 500 nM, equal to or less than about 400 nM, equal to or less than about 300 nM equal to or less than about 250 nM, equal to or less than about 200 nM, equal to or less than about 150 nM, equal to or less than about 100 nM, equal to or less than about 50 nM or equal to or less than about 10 nM, or any equivalent unit of measure (e.g., mol/L), as determined by an assay of methylation activity. The EZH2 inhibitor may exhibit an IC₅₀ value of equal to or less than any value defined herein. By way of example, the EZH2 inhibitor may be one or more of the following: GSK126, which exhibits an IC₅₀ of 9.9 nM as determined by an assay of methylation activity as described in McCabe et al (Nature, 2012, 492(7427), 108-112); GSK343, which exhibits an IC₅₀ of 4 nM as determined by an assay of methylation activity as described in Verma et al (ACS Med. Chem. Lett. 2012, 3, 12, 1091-1096); EPZ-6438, which exhibits an IC₅₀ of 2.5 nM as determined by an assay of methylation activity as described in Knutson et al (Proc Natl Acad Sci USA, 2013 May 7; 110(19):7922-7); CPI-1205, which exhibits an IC₅₀ of 2 nM as determined by an assay of methylation activity as described in Vaswani et al (J Med Chem, 2016, 59, 21, 9928-9941); and PF-06821497, which exhibits an IC₅₀ of 4 nM as determined by an assay of methylation activity as described in Kung et al (J Med Chem, 2018, February 8; 61(3):650-665).

The assay of methylation activity may be any suitable assay known in the art. An example of a suitable assay of methylation comprises methylation of H3 (histone 3) by 5 member PRC2 complex (Flag-EZH2, EED, SUZ12, AEBP2, RbAp48) in the presence of [³H]-SAM (tritiated SAM, S-adenosyl-L-[methyl-³H]methionine), monitored by detection of the tritiated histone product (histone N⁶-[methyl-³H]-L-lysine).

The inhibitor of EZH2 may be any small molecule EZH2 inhibitor known in the art. The term “small molecule” denotes a generally low molecular weight compound and includes organic and inorganic compounds. In general, a small molecule has a well-defined chemical formula with a single molecular weight. Preferably, a small molecule has a molecular weight of less than 3000 Daltons. More preferably, a small molecule has a molecular weight of less than 2000 Daltons. In some embodiments, the small molecule has a molecular weight of less than 1000 Daltons.

Inhibitors of EZH2 described in Duan et al (Journal of Hematology & Oncology, Volume 13, Article number: 104 (2020)); Shahabipour et al (Cancer Lett. 2017 Aug. 1; 400:325-335); Stazi et al (Expert Opinion on Therapeutic Patents, 27:7, 797-813, 2017); WO2011/140324 (US20130053397A1); WO2011/140325 (US20130053383A1); WO2012/142504 (US20120264734A1); WO2013/120104 (US20150011546A1); and WO2015/193765 (US20150361067A1) may be useful in the present invention. These references in their entirety are herein incorporated by reference.

In some embodiments, the EZH2 inhibitor is ursolic acid, which has the following structure:

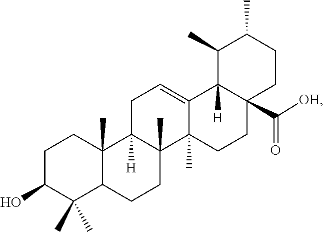

or a stereoisomer, a tautomer, a hydrate, a solvate, or a salt thereof, in particular a pharmaceutically acceptable salt, or a mixture of same.

In some embodiments, the inhibitor of EZH2 is a compound of formula (I):

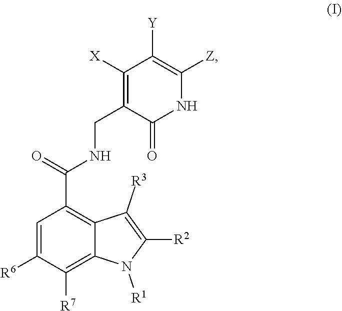

wherein
X and Z are selected independently from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halo, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, NR^{a}R^{b}, NR^{a}C(O)R^{b}, NR^{a}C(O)NR^{a}R^{b}, —NR^{a}C(O)OR^{a}, —NR^{a}SO₂R^{b}, NR^{a}SO₂NR^{a}R^{b}, NR^{a}NR^{a}R^{b}, NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};
Y is H or halo;
R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}. —CONR^{a}R^{b}, —CONR^{a}NR^{a}R^{b};
R² is hydrogen, (C₁-C₈)alkyl, trifluoromethyl, alkoxy, or halo, in which said (C₁-C₈)alkyl maybe substituted with one to two groups selected from: amino, and (C₁-C₃)alkylamino;
R⁷ is hydrogen, (C₁-C₃)alkyl, or alkoxy; R³ is hydrogen, (C₁-C₈)alkyl, cyano, trifluoromethyl, —NR^{a}R^{b}, or halo; R⁶ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, NR^{a}C(O)NR^{a}R^{b}, NR^{a}C(O)OR^{a}, NR^{a}SO₂R^{b}, NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, NR^{a}NR^{a}C(O)OR^{a}, —OR^{a}, —OC(O)R^{a}, —OC(O)NR^{a}R^{b};
   wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, NR^{a}C(O)R^{b}, —NR^{a}C(O)NR^{a}R^{b}, —NR^{a}C(O)OR^{a}, NR^{a}SO₂R^{b}, NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, NR^{a}R^{b}, NR^{a}C(O)R^{b}, NR^{a}C(O)NR^{a}R^{b}, NR^{a}C(O)OR^{a}, —NR^{a}SO₂R^{b}. NR^{a}SO₂NR^{a}R^{b}, —OR^{a}, —OC(O)R^{a}, and —OC(O)NR^{a}R^{b};
each R^{o} is independently (C₁-C₄)alkylamino, —NR^{a}SO₂R^{b}, —SOR^{a}, —SO₂R^{a}, —NR^{a}C(O)OR^{a}, —NR^{a}R^{b}, or —CO₂R^{a};
R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl)((C₁-C₄)alkyl);
or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;
or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;
or a stereoisomer, a tautomer, a hydrate, a solvate, or a salt thereof, in particular a pharmaceutically acceptable salt, or a mixture of same.

The EZH2 inhibitor, or the compound of formula (I), may be any compound disclosed in WO2011/140324 (US20130053397A1), including GSK126, or a stereoisomer, a tautomer, a hydrate, a solvate, or a salt thereof, in particular a pharmaceutically acceptable salt, or a mixture of same.

In some embodiments, the EZH2 inhibitor, or the compound of formula (I), is GSK126, which has the following structure:

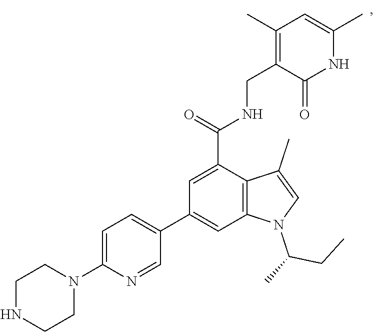 [SMILES: CC[C@H](C)n1cc(C)c2c(C(=O)NCc3c(C)cc(C)[nH]c3=O)cc(-c3ccc(N4CCNCC4)nc3)cc21]

or a stereoisomer, a tautomer, a hydrate, a solvate, or a salt thereof, in particular a pharmaceutically acceptable salt, or a mixture of same.

For compounds of formula (I), for avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

The term “alkyl” refers to a straight- or branched-chain hydrocarbon radical having the specified number of carbon atoms, so for example, as used herein, the terms “C₁-C₈alkyl” refers to an alkyl group having at least 1 and up to 8 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, and n-octyl and branched analogs of the latter 5 normal alkanes.

The term “alkoxy” as used herein means —O(C₁-C₈alkyl) including —OCH₃, —OCH₂CH₃ and —OC(CH₃)₃ and the like per the definition of alkyl above.

The term “alkylthio” as used herein is meant —S(C₁-C₈alkyl) including —SCH₃, —SCH₂CH₃ and the like per the definition of alkyl above.

The term “acyloxy” means —OC(O)C₁-C₈alkyl and the like per the definition of alkyl above.

The term “acylamino” means-N(H)C(O)C₁-C₈alkyl and the like per the definition of alkyl above.

The term “aryloxy” means —O(aryl), —O(substituted aryl), —O(heteroaryl) or —O(substituted heteroaryl).

The term “arylamino” means —NH(aryl), —NH(substituted aryl), —NH(heteroaryl) or —NH(substituted heteroaryl), and the like.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkyhylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

The term “haloalkyl” refers to an alkyl group group that is substituted with one or more halo substituents, suitably from 1 to 6 substituents. Haloalkyl includes trifluoromethyl.

When “cycloalkyl” is used it refers to a non-aromatic, saturated, cyclic hydrocarbon ring containing the specified number of carbon atoms. So, for example, the term “C₃-C₈cycloalkyl” refers to a non-aromatic cyclic hydrocarbon ring having from three to eight carbon atoms. Exemplary “C₃-C₈cycloalkyl” groups useful in the present invention include, but are not limited to, cyclopropyl, cyclobutyl, cyclopentyl, cyclohexyl, cycloheptyl and cyclooctyl.

The term “C₅-C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈heterocycloalkyl” is used, it means a non-aromatic heterocyclic ring containing the specified number of ring atoms being, saturated or having one or more degrees of unsaturation and containing one or more heteroatom substitutions independently selected from O, S and N. Such a ring may be optionally fused to one or more other “heterocyclic” ring(s) or cycloalkyl ring(s). Examples are given herein.

“Aryl” refers to optionally substituted monocyclic or polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl, and the like, as further illustrated herein.

“Heteroaryl” means an optionally substituted aromatic monocyclic ring or polycarbocyclic fused ring system wherein at least one ring complies with Hückel's Rule, has the specified number of ring atoms, and that ring contains at least one heteratom independently selected from N, O and S. Examples of “heteroaryl” groups are given herein.

The EZH2 inhibitor may be any compound disclosed in WO2011/140325 (US20130053383A1), including GSK343, or a stereoisomer, a tautomer, a hydrate, a solvate, or a salt thereof, in particular a pharmaceutically acceptable salt, or a mixture of same.

In some embodiments, the EZH2 inhibitor is GSK343, which has the following structure:

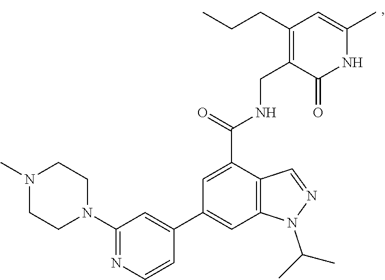

or a stereoisomer, a tautomer, a hydrate, a solvate, or a salt thereof, in particular a pharmaceutically acceptable salt, or a mixture of same. GSK-126 exhibits about >150-fold selectivity for EZH2 over EZH1

The EZH2 inhibitor may be any compound disclosed in WO2012/142504 (US20120264734A1), including EPZ-6438 (Tazemetostat), or a stereoisomer, a tautomer, a hydrate, a solvate, or a salt thereof, in particular a pharmaceutically acceptable salt, or a mixture of same.

In some embodiments, the EZH2 inhibitor is EPZ-6438 (Tazemetostat), which has the following structure:

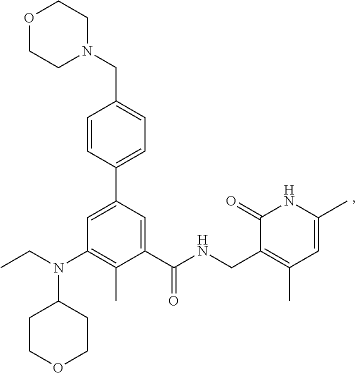 [SMILES: CCN(c1cc(-c2ccc(CN3CCOCC3)cc2)cc(C(=O)NCc2c(C)cc(C)[nH]c2=O)c1C)C1CCOCC1]

or a stereoisomer, a tautomer, a hydrate, a solvate, or a salt thereof, in particular a pharmaceutically acceptable salt, or a mixture of same. EPZ-6438 competitively binds to the S-adenosylmethionine (SAM) binding site of EZH2 and also non-competitively binds to the binding sites of peptide or nucleosome substrate. EPZ-6438 selectively inhibits EZH2, with selectivity of about 35-fold greater than EZH1.

The EZH2 inhibitor may be any compound disclosed in WO2013/120104 (US20150011546A1), including CPI-1205, or a stereoisomer, a tautomer, a hydrate, a solvate, or a salt thereof, in particular a pharmaceutically acceptable salt, or a mixture of same.

In some embodiments, the EZH2 inhibitor is CPI-1205, which has a the following structure:

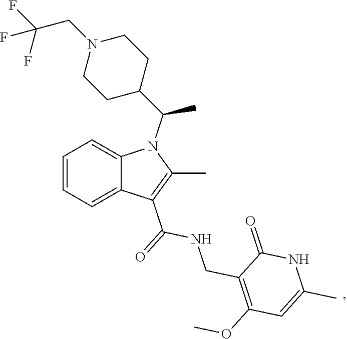 [SMILES: COc1cc(C)[nH]c(=O)c1CNC(=O)c1c(C)n([C@H](C)C2CCN(CC(F)(F)F)CC2)c2ccccc12]

or a stereoisomer, a tautomer, a hydrate, a solvate, or a salt thereof, in particular a pharmaceutically acceptable salt, or a mixture of same.

The EZH2 inhibitor may be any compound disclosed in WO2015/193765 (US20150361067A1), including PF-06821497, or a stereoisomer, a tautomer, a hydrate, a solvate, or a salt thereof, in particular a pharmaceutically acceptable salt, or a mixture of same.

In some embodiments, the EZH2 inhibitor is PF-06821497, which has the following structure:

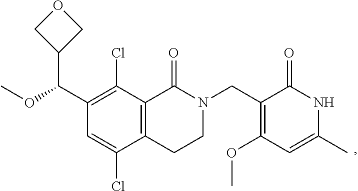

or a stereoisomer, a tautomer, a hydrate, a solvate, or a salt thereof, in particular a pharmaceutically acceptable salt, or a mixture of same.

The EZH2 inhibitor may be any compound disclosed in Shahabipour et al (Cancer Lett. 2017 Aug. 1; 400:325-335), including ursolic acid, triptolide and sulforaphane, or a stereoisomer, a tautomer, a hydrate, a solvate, or a salt thereof, in particular a pharmaceutically acceptable salt, or a mixture of same.

In some embodiments, the EZH2 inhibitor is triptolide, which has the following structure:

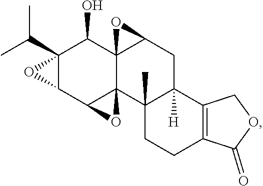

or a stereoisomer, a tautomer, a hydrate, a solvate, or a salt thereof, in particular a pharmaceutically acceptable salt, or a mixture of same.

In some embodiments, the EZH2 inhibitor is sulforaphane, which has the following structure:

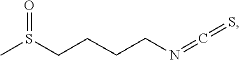

or a stereoisomer, a tautomer, a hydrate, a solvate, or a salt thereof, in particular a pharmaceutically acceptable salt, or a mixture of same.

In preferred embodiments, the inhibitor of EZH2 is selected from the group consisting of the following, or a stereoisomer, a tautomer, a hydrate, a solvate, or a salt thereof, in particular a pharmaceutically acceptable salt, or a mixture of same:

| Compound | CAS no | Chemical name | Chemical structure |
|---|---|---|---|
| Ursolic acid | 77-52-1 | 3β-Hydroxyurs-12-en-28-oic acid | 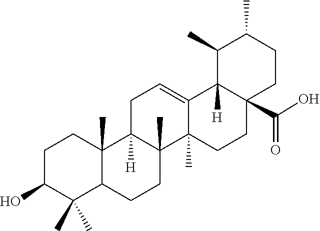 |
| GSK126 | 1346574-57-9 | N-[(1,2-Dihydro-4,6- dimethyl-2-oxo-3- pyridinyl)methyl]-3-methyl-1- [(1S)-1- methylpropyl]-6-[6-(1- piperazinyl)-3-pyridinyl]-1H- indole-4-carboxamide | 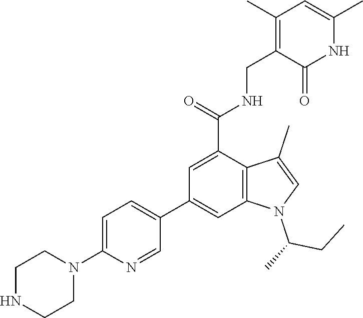 [SMILES: CC[C@H](C)n1cc(C)c2c(C(=O)NCc3c(C)cc(C)[nH]c3=O)cc(-c3ccc(N4CCNCC4)nc3)cc21] |
| GSK343 | 1346704-33-3 | N-[(1,2-Dihydro-6-methyl-2- oxo-4-propyl-3- pyridinyl)methyl]-1-(1- methylethyl)-6-[2-(4-methyl- 1-piperazinyl)-4-pyridinyl]- 1H-indazole-4- carboxamide | 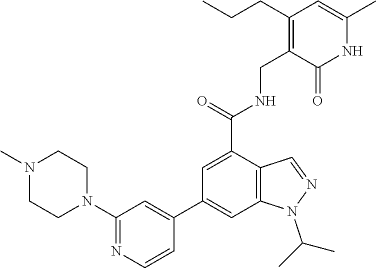 |
| EPZ-6438 (Tazemetostat) | 1403254-99-8 | N-[(1,2-Dihydro-4,6- dimethyl-2-oxo-3- pyridinyl)methyl]-5- [ethyl(tetrahydro- 2H-pyran-4-yl)amino]-4- methyl-4′-(4- morpholinylmethyl)[1,1′- biphenyl]-3- carboxamide | 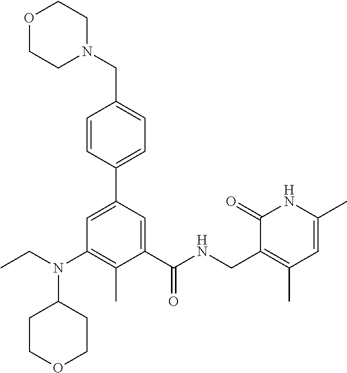 [SMILES: CCN(c1cc(-c2ccc(CN3CCOCC3)cc2)cc(C(=O)NCc2c(C)cc(C)[nH]c2=O)c1C)C1CCOCC1] |
| CPI-1205 | 1621862-70-1 | N-[(1,2-Dihydro-4-methoxy- 6-methyl-2-oxo-3- pyridinyl)methyl]-2-methyl-1- [(1R)-1-[1-(2,2,2- trifluoroethyl)-4- piperidinyl]ethyl]-1H-indole- 3-carboxamide | 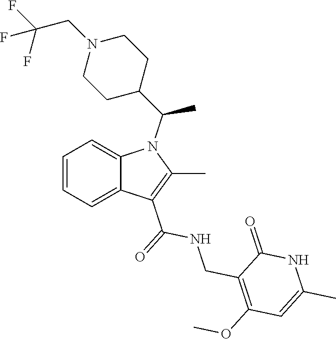 [SMILES: COc1cc(C)[nH]c(=O)c1CNC(=O)c1c(C)n([C@H](C)C2CCN(CC(F)(F)F)CC2)c2ccccc12] |
| PF-06821497 | 1844849-10-0 | (R)-5,8-Dichloro-7- (methoxy(oxetan-3- yl)methyl)-2-((4-methoxy-6- methyl-2-oxo-1,2- dihydropyridin-3-yl)methyl)- 3,4-dihydroisoquinolin- 1(2H)-one | 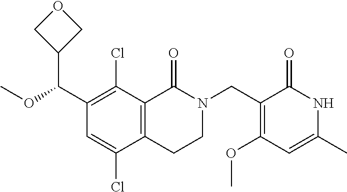 |
| Triptolide | 38748-32-2 | (3bS,4aS,5aS,6R,6aR,7aS, 7bS,8aS,8bS)- 3b,4,4a,6,6a,7a,7b,8b,9,10- Decahydro- 6-hydroxy-8b-methyl-6a-(1- methylethyl)trisoxireno[4b,5: 6,7:8a,9]phenanthro[1,2- c]furan-1(3H)-one | 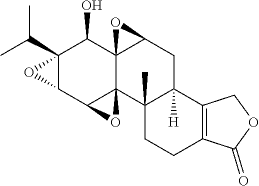 |
| Sulforaphane | 4478-93-7 | 1-Isothiocyanato-4- (methylsulfinyl)butane | 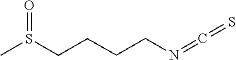 |

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds described herein may contain one or more asymmetric centre, depending upon the location and nature of the various substituents desired. Asymmetric carbon atoms is present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds described herein may contain sulphur atoms which are asymmetric, such as an asymmetric sulfoxide, of structure:

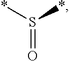

for example,
in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds described herein are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds described herein as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound described herein is achieved by any suitable state of the art method, such as chromatography, especially chiral chromatography, for example.

Further, the compounds described herein may exist as tautomers. The present invention includes all possible tautomers of the compounds of the present invention as single tautomers, or as any mixture of said tautomers, in any ratio.

Further, the compounds described herein may exist as N-oxides, which are defined in that at least one nitrogen of the compounds described herein is oxidised. The present invention includes all such possible N-oxides.

The present invention also relates to useful forms of the compounds described herein, such as metabolites, hydrates, solvates, prodrugs, salts, in particular pharmaceutically acceptable salts, and co-precipitates.

The compounds described herein may exist as a hydrate, or as a solvate, wherein the compounds described herein contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds described herein may exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

Examples of acid addition salts include, but are not limited to, hydrochlorides, hydrobromides, phosphates, nitrates, sulfates, salts of sulfamic acid, formates, acetates, propionates, citrates, D-gluconates, benzoates, 2-(4-hydroxybenzoyl)-benzoates, butyrates, salicylates, sulfosalicylates, lactates, maleates, laurates, malates, fumarates, succinates, oxalates, malonates, pyruvates, acetoacetates, tartarates, stearates, benzensulfonates, toluenesulfonates, methanesulfonates, trifluoromethansulfonates, 3-hydroxy-2-naphthoates, benzenesulfonates, naphthalinedisulfonates and trifluoroacetates.

Examples of salts with bases include, but are not limited to, lithium, sodium, potassium, calcium, aluminium, magnesium, titanium, meglumine, ammonium, salts optionally derived from NH₃ or organic amines having from 1 to 16 C-atoms such as e.g. ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylendiamine, N-methylpiperindine and guanidinium salts.

The salts include water-insoluble and, particularly, water-soluble salts.

The term “pharmaceutically acceptable salt” refers to a relatively non-toxic, inorganic or organic acid addition salt of a compound described herein. For example, see S. M. Berge, et al. “Pharmaceutical Salts,” J. Pharm. Sci. 1977, 66, 1-19.

A suitable pharmaceutically acceptable salt of the compounds described herein may be, for example, an acid-addition salt of a compound described herein bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound described herein which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the compounds described herein may be prepared by reaction of the compounds with the appropriate inorganic or organic acid via any of a number of known methods. Alternatively, alkali and alkaline earth metal salts of acidic compounds described herein are prepared by reacting the compounds with the appropriate base via a variety of known methods.

The present invention includes all possible salts of the compounds described herein as single salts, or as any mixture of said salts, in any ratio.

As used herein, when a compound is mentioned as a salt form with the corresponding base or acid, the exact stoichiometric composition of said salt form, as obtained by the respective preparation and/or purification process, is, in most cases, unknown. Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form. This applies analogously to cases in which synthesis intermediates or example compounds or salts thereof have been obtained, by the preparation and/or purification processes described, as solvates, such as hydrates with (if defined) unknown stoichiometric composition.

As used herein, the term “in vivo hydrolysable ester” is understood as meaning an in vivo hydrolysable ester of a compound described herein containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds described herein.

An in vivo hydrolysable ester of a compound described herein containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds described herein, either as single polymorph, or as a mixture of more than one polymorph, in any ratio.

### Applications

The insulin-producing cell, or the cell population comprising the insulin-producing cell, produced by a method as described herein, may be administered to an individual in need thereof.

Accordingly, in some embodiments of the method of producing an insulin-producing cell from a pancreatic exocrine cell, the method further comprises a step of administering the insulin-producing cell, or the cell population comprising the insulin-producing cell, to an individual.

In some embodiments, the pancreatic exocrine cell from which the insulin-producing cell is generated is obtained from the individual. Advantageously, since the the insulin-producing cell is derived from the individual's own cell, this may avoid the need for immunosuppresants when administering the insulin-producing cell to the individual.

The insulin-producing cell, or cell population comprising the insulin-producing cell, may be useful for administering to an individual with a disease involving dysfunctional insulin production. Accordingly, in some embodiments, the individual being administered the insulin-producing cell, or the cell population comprising the insulin-producing cell, has diabetes mellitus, for example type 1 diabetes, type 2 diabetes or gestational diabetes, or be pre-diabetic.

The present invention also provides a method for preventing or treating a disease involving dysfunctional insulin production, the method comprising:
administering a cell population comprising an insulin-producing cell as described herein or produced by a method as described herein, to an individual in need thereof,
thereby preventing or treating the disease.

In another aspect, the present invention provides the use of an insulin-producing cell, or a cell population comprising an insulin-producing cell, as described herein or produced by a method as described herein, for preventing or treating a disease involving dysfunctional insulin production in an individual.

In another aspect, the present invention provides the use of an insulin-producing cell, or a cell population comprising an insulin-producing cell, as described herein or produced by a method as described herein, in the manufacture of a composition (or a medicament) for preventing or treating a disease involving dysfunctional insulin production in an individual.

In another aspect, the present invention provides an insulin-producing cell, or a cell population comprising an insulin-producing cell, as described herein or produced by a method as described herein, for use in preventing or treating a disease involving dysfunctional insulin production in an individual.

Diseases involving dysfunctional insulin production include diabetes mellitus and pre-diabetes.

Diabetes mellitus, commonly known as diabetes, is a metabolic disease that causes high blood sugar. There are several different types of diabetes, including type type 1 diabetes, type 2 diabetes and gestational diabetes.

Type 1 diabetes (T1 D) refers to a chronic autoimmune disease that selectively destroys the insulin-producing β-cells in the pancreas. Type 1 diabetes is not linked to modifiable lifestyle factors. Typical symptoms include excessive thirst, frequent urination, unexplained weight loss, feeling extreme hunger, fatigue and weakness, mood swings, itching and skin infections, and visual disturbances such as blurred vision. The main diagnostic test is taking a blood test to measure glucose, with fasting or at other times of the day. Tests include fasting blood glucose test, random blood glucose test, and oral glucose tolerance test.

Type 2 diabetes (T2D) refers to a condition in which the body becomes resistant to the normal effects of insulin and gradually loses the capacity to produce enough insulin in the pancreas. The condition has strong genetic and family-related (non-modifiable) risk factors and is also often associated with modifiable lifestyle risk factors. Typical symptoms include excessive thirst, frequent urination, unexplained weight loss, feeling extreme hunger, fatigue and weakness, mood swings, itching and skin infections, and visual disturbances such as blurred vision. The main diagnostic test is taking a blood test to measure glucose. Tests include fasting blood glucose test, oral glucose tolerance test and haemoglobin A1C test.

Gestational diabetes is diabetes that occurs during pregnancy. Insulin-blocking hormones produced by the placenta cause gestational diabetes. Gestational diabetes usually goes away after delivery, but can raise the risk of the invidivual developing type 2 diabetes in the future. Symptoms are typically mild and may include excessive thirst, frequent urination, fatigue, blurred vision, and yeast infections. Gestational diabetes is typically diagnosed by a pregnancy oral glucose tolerance test.

Pre-diabetes (also referred to as intermediate diabetes) refers to a condition where an individual's blood sugar is higher than normal, but not high enough for a type 2 diabetes diagnosis. Individuals with pre-diabetes (also referred to as intermediate diabetics) are defined as typically having fasting plasma glucose (FPG) levels of ≥100 mg/dl (5.6 mmol/1) but <126 mg/dl (7.0 mmol/1). Pre-diabetes is often associated with modifiable lifestyle risk factors. The condition has no clear symptoms; a possible sign of pre-diabetes is darkened skin on certain parts of the body including on the neck, armpits and groin. Diagnostic tests include fasting blood glucose test, oral glucose tolerance test and haemoglobin A1C test. Individuals with pre-diabetes are considered high-risk in developing diabetes.

In some embodiments, the individual is identified as having diabetes mellitus, for example type 1 diabetes, type 2 diabetes or gestational diabetes, or pre-diabetes.

In some embodiments, the diabetes or pre-diabetes may be associated with one or more of the following: endocrinopathy (a disease associated with dysfunction of an endocrine gland) including endocrinopathy associated with administration of a hormone (e.g. cortisol), a disease of the exocrine pancreas (e.g. due to infection, trauma), drug-induced diabetes (e.g., diabetes associated with administration of α-interferon), and a genetic disorder (e.g., Down syndrome).

In some embodiments, the pancreatic exocrine cell from which the insulin-producing cell of the cell population is generated is from pancreatic tissue obtained from the individual. As shown in the Examples, the methods and uses of the invention allow for production of insulin-producing cells from pancreatic tissue obtained from non-diabetic and diabetic donors. Advantageously, since the the insulin-producing cell is derived from the individual's own cell, this may avoid the need for immunosuppresants when administering the insulin-producing cell to the individual.

In some embodiments, the cell population comprising the insulin-producing cell is delivered to the pancreas of the individual. The cell population may be delivered by any suitable method known in the art. For example, the cell population may be delivered to the pancreas by injecting the cell population into the portal vein of the liver of the individual.

In another aspect, the present invention provides a method for preventing or treating a disease involving dysfunctional insulin production, the method comprising:
administering an inhibitor of EZH2 to an individual in need thereof such that the inhibitor of EZH2 contacts a pancreatic exocrine cell of the individual for a sufficient time and under conditions to allow generation of an insulin-producing cell from the pancreatic exocrine cell,
thereby preventing or treating the disease in the individual.

The present invention also provides the use of an inhibitor of EZH2 for preventing or treating a disease involving dysfunctional insulin production in an individual, wherein the inhibitor of EZH2 contacts a pancreatic exocrine cell of the individual for a sufficient time and under conditions to allow generation of an insulin-producing cell from the pancreatic exocrine cell.

The present invention also provides the use of an inhibitor of EZH2 in the manufacture of a composition (or a medicament) for preventing or treating a disease involving dysfunctional insulin production in an individual, wherein the inhibitor of EZH2 contacts a pancreatic exocrine cell of the individual for a sufficient time and under conditions to allow generation of an insulin-producing cell from the pancreatic exocrine cell.

The present invention also provides an inhibitor of EZH2 for use in preventing or treating a disease involving dysfunctional insulin production in an individual, wherein the inhibitor of EZH2 contacts a pancreatic exocrine cell of the individual for a sufficient time and under conditions to allow generation of an insulin-producing cell from the pancreatic exocrine cell.

In another aspect, the present invention provides a pharmaceutical composition for use in treating or preventing a disease involving dysfunctional insulin production, the pharmaceutical composition comprising an inhibitor of EZH2 and a pharmaceutically acceptable diluent, excipient or carrier. In one embodiment, the only active ingredient present in the composition is an inhibitor of EZH2.

The present invention also provides a pharmaceutical composition for use in treating or preventing a disease involving dysfunctional insulin production, the composition comprising an inhibitor of EZH2 as an active ingredient, and a pharmaceutically acceptable diluent, excipient or carrier. In one embodiment, the only active ingredient present in the composition is an inhibitor of EZH2.

The present invention also provides a pharmaceutical composition for use in treating or preventing a disease involving dysfunctional insulin production, the composition comprising an inhibitor of EZH2 as a main ingredient, and a pharmaceutically acceptable diluent, excipient or carrier. In one embodiment, the only active ingredient present in the composition is an inhibitor of EZH2.

The disease involving dysfunctional insulin production may be diabetes mellitus, for example type 1 diabetes, type 2 diabetes or gestational diabetes, or pre-diabetes.

In some embodiments, the individual is identified as having diabetes mellitus, for example type 1 diabetes, type 2 diabetes or gestational diabetes, or pre-diabetes.

The inhibitor of EZH2 may be any EZH2 inhibitor as described herein. The EZH2 inhibitor may be administered systemically or directly to the site of disease. The EZH2 inhibitor may be formulated for oral administration.

As used herein, the terms “treatment” or “treating” of a subject include the application or administration of an insulin-producing cell produced by a method as described herein (or a cell population comprising an insulin-producing cell produced by a method as described herein) to a subject, or application or administration of an EZH2 inhibitor described herein to a subject (or to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, the terms “prevention” or “preventing” are intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are known in the art.

The existence of, or improvement in, treatment or prevention of diabetes and pre-diabetes may be by any clinically or biochemically relevant method known in the art. Examples of relevant measures include blood glucose tests including as those described herein. A relevant method include measurement of blood glucose, blood pressure, cholesterol levels and body weight. For example, a positive response to treatment may be an improvement (e.g., reduction) in blood glucose levels or controlling blood glucose levels. The improvement or treatment may be determined directly from the subject, or a sample or biopsy therefrom. The sample may be a blood test. The sample or biopsy may be pancreatic tissue or pancreatic cells therefrom.

### Administration of EZH2 Inhibitor

In some embodiments, the method for preventing or treating a disease involving dysfunctional insulin production comprises administering a therapeutically effective amount of an EZH2 inhibitor as described herein or a pharmaceutical composition as described herein.

As used herein, the term “therapeutically effective amount” is generally intended to refer to an amount of an active agent, such as a compound described herein, that (i) treats the disease involving dysfunctional insulin production, (ii) attenuates, ameliorates or eliminates one or more symptoms of the disease involving dysfunctional insulin production, or (iii) delays the onset of one or more symptoms of the disease involving dysfunctional insulin production as described herein. The exact dose will depend on the purpose of the treatment, and will be ascertainable by one skilled in the art using known techniques. As is known in the art and described herein, adjustments for systemic versus localized delivery, age, body weight, general health, sex, diet, time of administration, drug interaction and the severity of the condition may be necessary, and would be ascertainable with routine experimentation by those skilled in the art.

Suitable dosages of a compound or pharmaceutical composition described herein will vary depending on the specific disease involving dysfunctional insulin production to be treated and/or the subject being treated. It is within the ability of a skilled physician to determine a suitable dosage, for example by commencing with a sub-optimal dosage and incrementally modifying the dosage to determine an optimal or useful dosage. Alternatively, to determine an appropriate dosage for treatment, data from cell culture assays or animal studies may be used, wherein a suitable dose is within a range of circulating concentrations that include the ED₅₀ of the active compound with little or no toxicity. The dosage may vary within this range depending upon the dosage form employed and the route of administration utilized. A therapeutically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

Typically, a therapeutically effective dosage is formulated to contain a concentration (by weight) of at least about 0.1% up to about 50% or more of the compound described herein, and all combinations and sub-combinations of ranges therein. The pharmaceutical composition as described herein can be formulated to contain a compound described herein or a pharmaceutically acceptable salt or solvent thereof in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 1 6, 1 5, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue in the subject.

The compound or pharmaceutical composition described herein may be administered, or formulated for administration by, any route described herein. As used herein, the term “administered” means administration of a therapeutically effective dose of the compound described herein to the subject. As used herein, the term “formulated for administration” means a therapeutically effective dose of the compound described herein is formulated in such a way that is suitable for the route of administration. In preferred embodiments, the compound (or pharmaceutical composition) is administered orally or parenterally, more preferably orally. In other preferred embodiments, the compound (or pharmaceutical composition) is formulated for oral administration or parenteral administration, more preferably for oral administration.

In embodiments where the compound (or pharmaceutical composition) is orally administered to a subject or formulated for oral administration to a subject, the therapeutically effective amount of a compound may correspond to preferably between about 1 to about 50 mg/kg, or between about 1 to 25 mg/kg, or between about 1 to about 10 mg/kg, between about 5 to about 25 mg/kg, or between about 10 to about 20 mg/kg.

The frequency of administration of a compound or pharmaceutical composition described herein may be once daily, twice daily or three times daily. The treatment period may be for the duration of the detectable disease.

Pharmaceutical compositions may be formulated for any appropriate route of administration including, for example, oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use, especially oral use, are preferred. Suitable oral forms include, for example, tablets, troches, lozenges, aqueous or oily suspensions, dispersible powders or granules, emulsions, hard or soft capsules, or syrups or elixirs. Within yet other embodiments, compositions provided herein may be formulated as a lyophilisate.

The various dosage units are each preferably provided as a discrete dosage tablet, capsules, lozenge, dragee, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydropropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such formulations may also comprise one or more demulcents, preservatives, flavouring agents and/or colouring agents.

Compounds described herein may be formulated for local or topical administration. Formulations for topical administration typically comprise a topical vehicle combined with active agent(s), with or without additional optional components.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, and sprays. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylceilulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used.

A pharmaceutical composition may be formulated as inhaled formulations, including sprays, mists, or aerosols. This may be particularly preferred for treatment of pulmonary fibrosis. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Pharmaceutical compositions may also be prepared in the form of suppositories such as for rectal administration. Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations such as a capsule that creates a slow release of modulator following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of modulator release. The amount of modulator contained within a sustained release formulation depends upon, for example, the site of implantation, the rate and expected duration of release and the nature of the condition to be treated or prevented.

In another aspect, the present invention relates to a composition comprising at least one pancreatic exocrine cell and at least one inhibitor of EZH2. The pancreatic exocrine cell and the EZH2 inhibitor may be any as described herein.

Also provided herein is a kit or article of manufacture including one or more inhibitors of EZH2 as described herein, or a pharmaceutically acceptable salt, polymorph or prodrug thereof and/or pharmaceutical composition as described above.

In some embodiments, the kit includes:
a container holding a therapeutic composition in the form of one or more inhibitors of EZH2 as described herein, or a pharmaceutically acceptable salt, polymorph or prodrug thereof or pharmaceutical composition;
a label or package insert with instructions for use.

Preferably, the present invention provides a kit when used in a method of the invention described herein. In some embodiments, there is provided a kit for use in producing an insulin-producing cell as described herein. In some embodiments, there is provided a kit for use in preventing or treating a disease involving dysfunctional insulin production.

The label or package insert may indicate that the therapeutic composition is used for in preventing or treating a disease involving dysfunctional insulin production. In one embodiment, the label or package insert includes instructions for use and indicates that the therapeutic or prophylactic composition can be used to prevent or treat a disease involving dysfunctional insulin production as described herein. In some embodiments, the kit comprises instructions for use in producing an insulin-producing cell from a pancreatic exocrine cell according to the methods as described herein.

It will be understood, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient), and the severity of the particular disorder undergoing therapy.

### EXAMPLES

The invention will be further described by way of non-limiting examples. It will be understood by persons skilled in the art of the invention that modifications may be made without departing from the spirit and scope of the invention.

The invention discloses several structurally distinct molecules, for example GSK126, EPZ-6438, Ursolic acid and triptolide, which are used to produce an insulin-producing cell by the described methods. These molecules although structurally dissimilar, have different modes of action, different affinities and inhibitory constants, and different selectivity for EZH2 but all share the same property of being inhibitors of EZH2. The results described below show that any EZH2 inhibitor can produce an insulin-producing cell from a pancreatic exocrine cell.

Human pancreatic ductal epithelial cells were stimulated with the EZH2 inhibitors GSK-126, EPZ6438, and Triptolide using a 2- and 7-day protocol to determine their influence on the expression core endocrine development marker NGN3, as well as β-cell markers insulin, MAFA, and PDX1. Chromatin immunoprecipitation studies show a close correspondence of pharmacological EZH2 inhibition with reduced H3K27me3 content of the core genes, NGN3, MAFA and PDX1. Consistent with the reduction of H3K27me3 by pharmacological inhibition of EZH2, the inventors observed measurable immunofluorescence staining of insulin protein and glucose sensitive insulin response.

### Example 1. Effect of GSK126 on Pancreatic Cells from Non-Diabetic and Type 1 Diabetic Donors

### Materials and Methods

### Human Samples

Rapid harvesting of cadaveric pancreatic tissues was obtained with informed consent from next of kin, from heart-beating, brain-dead donors, with research approval from the Human Research Ethics Committee at St Vincent's Hospital, Melbourne. Pancreas from individuals without and with diabetes, islet, acinar and ductal samples were obtained as part of the research consented tissues through the National Islet Transplantation Program (at Westmead Hospital, Sydney and the St Vincent's Institute, Melbourne, Australia), HREC Protocol number: 011/04. The donor characteristics of islet cell donor isolations are presented in Table 1.

TABLE 1
Clinical characteristics of type 1 diabetic and non-diabetic donors

| | Donor 1 | Donor 2 | Donor 3 |
|---|---|---|---|
| Age | 58 | 42 | 13 |
| Sex | Male | Female | Female |
| BMI | 28.3 | 29 | 18.7 |
| Diabetes | Non-diabetic | Non-diabetic | Type 1 |
| Diabetes duration | N/A | N/A | 4.4 years |
| HbA1c | N/A | N/A | 7.7 |

### Preparation and Culture of Isolated Human Pancreatic Cells

Islets were purified by intraductal perfusion and digestion of the pancreases with collagenase AF-1 (SERVA/Nordmark, Germany) (Ricordi C et al. Automated method for isolation of human pancreatic islets. Diabetes 37, 413-420 (1988)), followed by purification using Ficoll density gradients (Barbaro B, et al. Improved human pancreatic islet purification with the refined UIC—UB density gradient. Transplantation 84, 1200-1203 (2007)). Purified islets, from low density gradient fractions, and acinar/ductal tissue, from high density fractions, were cultured in Miami Media 1A (Mediatech/Corning 98-021, USA) supplemented with 2.5% human serum albumin (Australian Red Cross, Melbourne, VIC, Australia), in a 37° C., 5% CO₂ incubator.

### Gene Expression Analysis

Total RNA from human ex vivo pancreatic cells was isolated using TRIzol (Invitrogen) and RNeasy Kit (QIAGEN) including a DNase treatment. First-strand cDNA synthesis was performed using a high-capacity cDNA Reverse Transcription Kit (Applied Biosystems) according to the manufacturer's instructions. cDNA primers were designed using oligoperfect designer (Thermo Fisher Scientific), as shown in Table 2 and Table 3. Briefly, quantitative RT-PCR analyses were undertaken using the PrecisionFast 2× qPCR Master Mix (Primerdesign) and primers using Applied Biosystems 7500 Fast Real-Time PCR System. Each qPCR reaction contained: 6.5 μl qPCR Master Mix, 0.5 μl of forward and reverse primers, 3.5 μl H₂O and 2 μl of previously synthesised cDNA, diluted 1/20. Expression levels of specific genes were tested and normalised to 18s ribosomal RNA housekeeping gene.

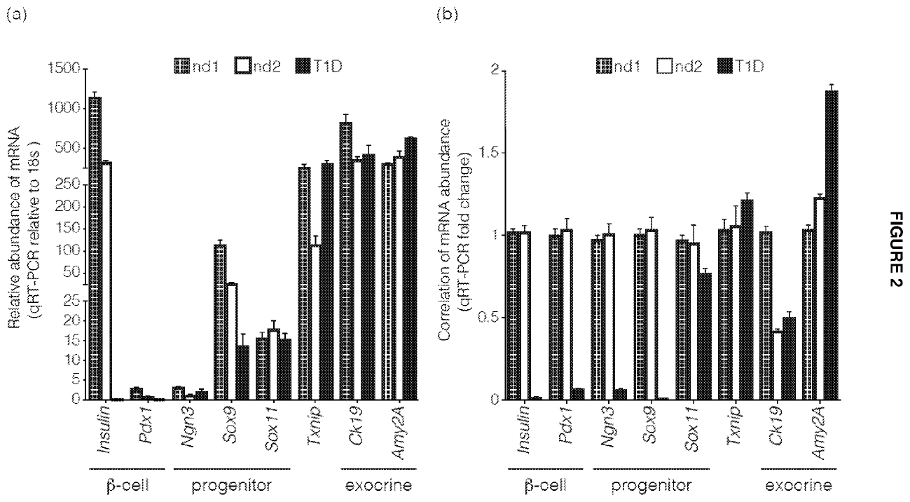

TABLE 2
Human primer sequences for mRNA expression

| Primer Name | Sequence | SEQ ID No. |
|---|---|---|
| NGN3 cDNA F | TCTCTATTCTTTTGCGCCGG | 1 |
| | | (used in experiments |
| | | to generate data in |
| | | FIGs. 2, 4, 7, 8 and |
| | | 9) |
| NGN3 cDNA R | CTTGGACAGTGGGCGCAC | 2 |
| | | (used in experiments |
| | | to generate data in |
| | | FIGs. 2, 4, 7, 8 and |
| | | 9) |
| SOX9 cDNA F | AGGAAGCTCGCGGACCAGTAC | 3 |
| SOX9 cDNA R | GGTGGTCCTTCTTGTGCTGCAC | 4 |
| INS cDNA F | ACGAGGCTTCTTCTACACACCC | 5 |
| | | (used in experiments |
| | | to generate data in |
| | | FIGs. 2, 4, 7, 8 and |
| | | 9) |
| INS cDNA R | TCCACAATGCCACGCTTCTGCA | 6 |
| | | (used in experiments |
| | | to generate data in |
| | | FIGs. 2, 4, 7, 8 and |
| | | 9) |
| PDX1 cDNA F | GAAGTCTACCAAAGCTCACGCG | 7 |
| PDX1 cDNA R | GGAACTCCTTCTCCAGCTCTAG | 8 |
| CK19 cDNA F | AGCTAGAGGTGAAGATCCGCGA | 9 |
| CK19 cDNA R | GCAGGACAATCCTGGAGTTCTC | 10 |
| AMY2A cDNA F | GATAATGGGAGCAACCAAGTGGC | 11 |
| AMY2A cDNA R | CAGTATGTGCCAGCAGGAAGAC | 12 |
| TXNIP cDNA F | ACGCTTCTTCTGGAAGACCA | 13 |
| TXNIP cDNA R | CGGTCAAGAAAAGCCTTCAC | 14 |
| MAFA cDNA F | GCTTCAGCAAGGAGGAGGTCAT | 15 |
| MAFA cDNA R | TCTGGAGTTGGCACTTCTCGCT | 16 |
| NKX6.1 cDNA F | CCTATTCGTTGGGGATGACAGAG | 17 |
| NKX6.1 cDNA R | TCTGTCTCCGAGTCCTGCTTCT | 18 |
| SOX11 cDNA F | GCTGAAGGACAGCGAGAAGATC | 19 |
| SOX11 cDNA R | GGGTCCATTTTGGGCTTTTTCCG | 20 |
| Ki67 cDNA F | GAAAGAGTGGCAACCTGCCTTC | 21 |
| Ki67 cDNA R | GCACCAAGTTTTACTACATCTGCC | 22 |
| IGF2 cDNA F | GCAGAGGAGTGTCCGGCAG | 23 |
| IGF2 cDNA R | GGATTCCCATTGGTGTCTGGAA | 24 |
| IGF2 AS cDNA F | CTGCCTAGAGCTCCCTCTTTC | 25 |
| IGF2 AS cDNA R | GCCTGTCCAACAGAAGGGTC | 26 |
| 18s cDNA F | CAGCCACCCGAGATTGAGCA | 27 |
| 18s cDNA R | TAGTAGCGACGGGCGGTGTG | 28 |
| H3F3A cDNA F | ACAAAAGCCGCTCGCAAGAGTG | 29 |
| H3F3A cDNA R | TTTCTCGCACCAGACGCTGGAA | 30 |
| INS hnRNA cDNA F | GAGATGGGGAAGATGCTGGG | 31 |
| INS hnRNA cDNA R | GGAGGACACAGTCAGGGAGA | 32 |
| EZH2 cDNA F | TCCTTTTCATGCAACACCCAACACT | 33 |
| EZH2 cDNA R | TCCAAATGCTGGTAACACTGTGGTC | 34 |
| EP300 cDNA F | GCAGTGTGCCAAACCAGATG | 35 |
| EP300 cDNA R | GGGTTTGCCGGGGTACAATA | 36 |
| KAT2 cDNA F | ATTCTGCAGGGGCCGAGCCT | 37 |
| KAT2 cDNA R | ATCACACGGAGCCGCTTGGC | 38 |
| BRG1 cDNA F | GCTCATGGCTGAAGATGAGG | 39 |
| BRG1 cDNA R | CAGGCGCTTGTCCTTCTTC 40 | 40 |

TABLE 3
Human primer sequences for ChIP

| | | SEQ |
|---|---|---|
| | | ID |
| Primer Name | Sequence | No. |
| PDX1 PromR1 F | ACGTTTCTGCAAAGCTGTCTAGTT | 41 |
| PDX1 PromR1 R | GGCTTCAAACCATTCAGTAACTTC | 42 |
| PDX1 PromR2 F | TGGCTGTGAACAAACTTCATAAAT | 43 |
| PDX1 PromR2 R | CACCGTGGCTTAAAAGTTTCTATT | 44 |
| NGN3 PromR1 F | TTGCTCCTAGCCTATCTTTCCTTA | 45 |
| NGN3 PromR1 R | CTTTAGAATTCCTGGACCCTTCTC | 46 |
| NGN3 PromR2 F | CTTCTGGTCGCCAAGTTCAG | 47 |
| NGN3 PromR2 R | AGCAGATAAAGCGTGCCAAG | 48 |
| INS-IGF2 PromR1 F | GGGAACATAGAGAAAGAGGTCTCA | 49 |
| INS-IGF2 PromR1 R | AATTAATCTCAGCTTCCCCCTAAC | 50 |
| INS PromR2 F | TTTATAGTCTCAGAGCCCATCTCC | 51 |
| INS PromR2 R | CCCCTGGTTAAGACTCTAATGACC | 52 |
| INS PromR3 F | GACCTCATCTTCCGTGTGATCT | 53 |
| INS PromR3 R | CAGATGACACTATGGGGGTGAT | 54 |

### Histone Extraction and Quantitative Immunoblotting

Trimethylation of Histone H3K27 (1H3K27me3) and acetylation of H3K27 (H3K27ac) protein signals were quantified in human pancreatic ductal epithelial cells (AddexBio) by the LI-COR Odyssey assay. The cells were treated with 5 or 10 μM of GSK126 (S7061, Selleckchem) for 48 hours and histones prepared using an acid extraction method. Acid histone extraction and immunoblotting were performed as described previously (Kaipananickal H, et al. Targeting Treatment Refractory NET by EZH2 Inhibition in Postural Tachycardia Syndrome. Circ Res 126, 1058-1060 (2020)). Protein concentrations were determined using Coomassie Reagent (Sigma) with BSA as a standard. Equal amounts (3 μg) of acid extract were separated by Nu-PAGE (Invitrogen), transferred to a PVDF membrane (Immobilon-FL; Millipore) and then probed with antibodies against H3K27me3 (07-449, Millipore), H3K27ac (ab4729, Abcam) and total histone H3 (14269, Cell Signaling Technology). Protein blotting signals were quantified by an infrared imaging system (Odyssey; LI-COR). H3K27me3, H3K27ac signals were quantified using total histone H3 signal as a loading control.

### Chromatin Immunoprecipitation

Chromatin immunoprecipitation assays in human exocrine cells were performed previously described (Rafehi H, et al. Vascular histone deacetylation by pharmacological HDAC inhibition. Genome Res 24, 1271-1284 (2014); Pirola L, et al. Genome-wide analysis distinguishes hyperglycemia regulated epigenetic signatures of primary vascular cells. Genome Res 21, 1601-1615 (2011)). Cells were fixed for 10 minutes with 1% formaldehyde and quenched for 10 mins with glycine (0.125 M) solution. Fixed cells were resuspended in sodium dodecyl (lauryl) sulfate (SDS) lysis buffer (1% SDS, 10 mM EDTA, 50 mM Tris-HCl pH 8.1) including a protease inhibitor cocktail (Roche Diagnostics GmBH, Mannheim, Germany) and homogenized followed by incubation on ice for 5 minutes. Soluble samples were sonicated to 200-600 bp and chromatin was resuspended in ChIP Dilution Buffer (0.01% SDS, 1.1% Triton X-100, 1.2 mM EDTA, 16.7 mM Tris-HCl pH 8.0 and 167 mM NaCl) and 20 μl of Dynabeads® Protein A (Invitrogen, Carlsbad, CA, USA) was added and pre-cleared. H3K27me3 antibody was used for immunoprecipitation of chromatin and incubated overnight at 4° C. (Khurana I, et al. SAHA attenuates Takotsubo-like myocardial injury by targeting an epigenetic Ac/Dc axis. Signal Transduct Target Ther 6, 159 (2021)). Immunoprecipitated DNA were collected by magnetic isolation, washed low salt followed by high salt buffers and eluted with 0.1 M NaHCO₃ with 1% SDS. Protein-DNA cross-links were reversed by adding Proteinase K (Sigma, St. Louis, MO, USA) and incubation at 62° C. for 2 hours. DNA was recovered using a Qiagen MinElute column (Qiagen Inc., Valencia, CA, USA). H3K27me3 content at the promoters of the INS, INS-IGF2, NGN3 and PDX1 genes were assessed by qPCR using primers designed from the integrative ENCODE resource (Zhang J, et al. An integrative ENCODE resource for cancer genomics. Nat Commun 11, 3696 (2020)).

### Immunohistochemistry

Insulin and glucagon localization in human islets were assessed using paraffin sections (5 μm thickness) of human pancreas tissue fixed in 10% neutral-buffered formalin and stained with hematoxylin and eosin (H&E) or prepared for immunohistochemistry. Insulin and glucagon were detected using Guinea Pig anti-insulin ( 1/100, DAKO) or mouse anti-glucagon ( 1/50) mAbs (polyclonal Abs, Sigma-Aldrich).

### Induction of Ex Vivo Human Pancreatic Progenitors

For pharmacological inhibition of EZH2, human pancreatic exocrine cells were kept untreated or stimulated with 10 mM GSK126 (S7061, Selleckchem) at a cell density of 1×10⁵ per well for 24 hr. After 24 hr of treatment, fresh Miami Media was added to the cells, which were treated again with 10 mM GSK126, and cultured for a further 24 hr. All cell incubations were performed in Miami Media 1A (Mediatech/Corning 98-021, USA) supplemented with 2.5% human serum albumin (Australian Red Cross, Melbourne, VIC, Australia), in a cell culture incubator at 37° C. in an atmosphere of 5% CO₂ for 48 hr using non-treated 6 well culture plates (Corning).

### Results

The effect of GSK126 on Nkx6.1, MafA, Pdx1, Ngn3, Sox1 land Sox9 was investigated using ex vivo human pancreatic tissues from three donors, and the effect of GSK126 on β-cell regeneration was assessed by an increase in insulin (Ins) gene expression.

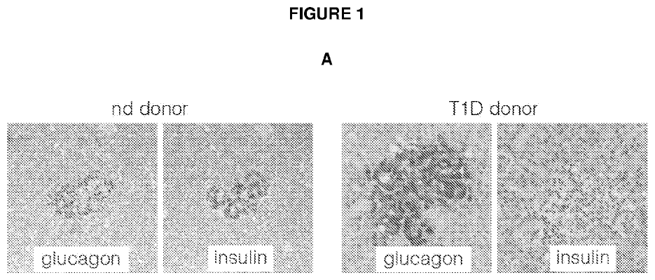

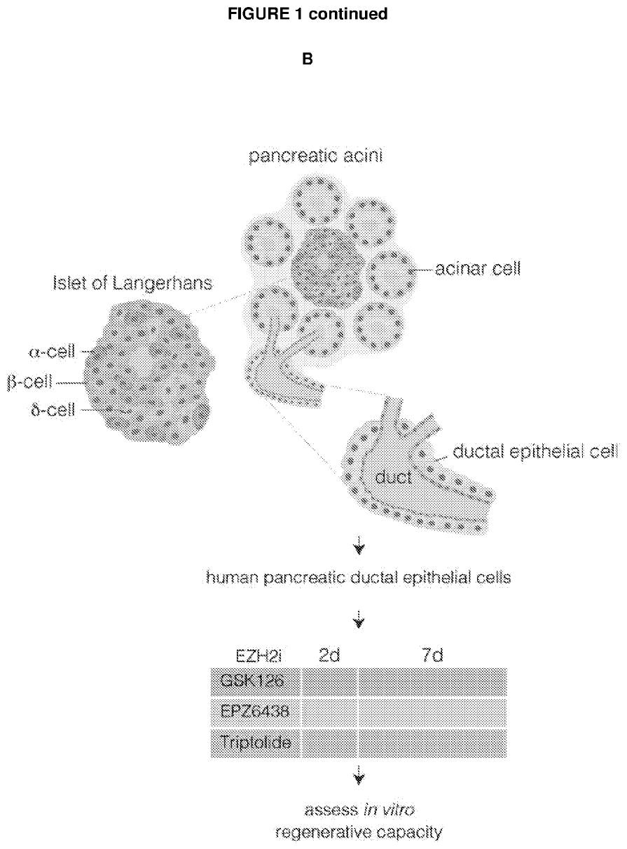

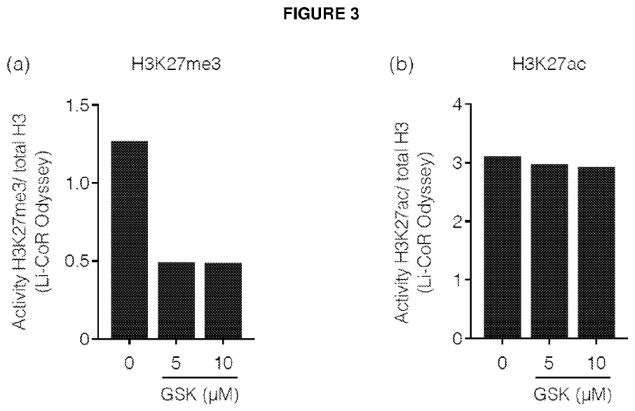

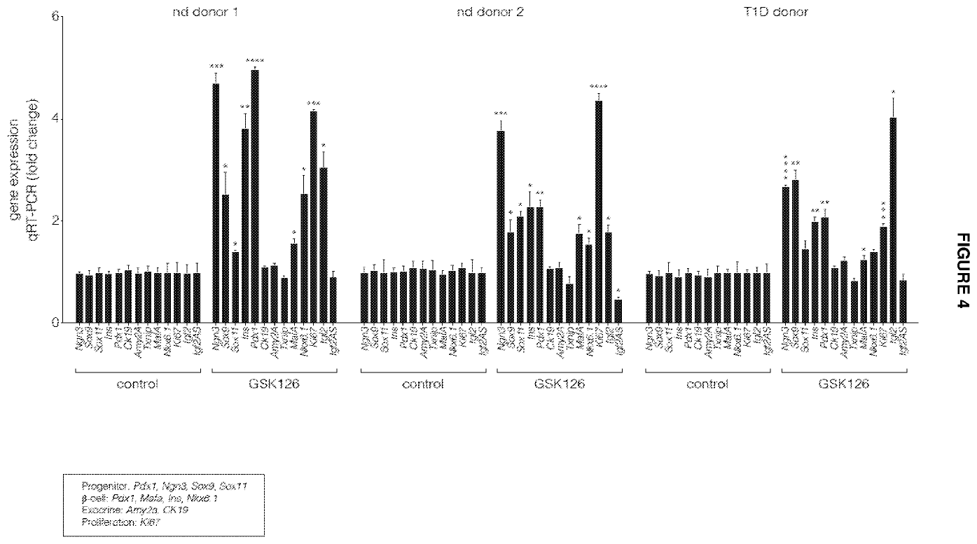

Pancreatic specimens were stained with H&E and immunostained for glucagon and insulin from a non-diabetic and a type 1 diabetes (T1 D) donor (FIG. 1). Insulin and glucagon expression are indicated by the staining in human islets. Note the complete absence of insulin in the T1 D donor. Insulin mRNA was present in the non-diabetic donor, in contrast to its absence in the T1 D donor, as expected. The expression of progenitor and embryogenic markers Pdx1, Ngn3, Sox9 and Sox11, as well as specific β-cell markers Pdx1, Ins, Nkx6.1, and MafA mRNA levels using qRT-PCR were mirrored in naïve pancreatic exocrine cells using the same non-diabetic and T1 D donors (FIG. 2). Li—COR analyses confirms GSK126 selectively reduced H3K27me3 levels (FIG. 3a) and failed to influence H3K27ac content (FIG. 3b).

FIG. 4 illustrates the effects of pharmacological inhibition using GSK126 on pancreatic exocrine cells from two non-diabetic donors and a type 1 diabetic donor. When stimulated with GSK126, the hallmark genes responsible for the maintenance of pancreatic progenitor identity Pdx1, Ngn3, and Sox9 were restored, and significant expression of the Ins gene was observed in the non-diabetic and the T1 D donors. These results may suggest that EZH2 inhibition can restore exocrine multipotency, even in chronic disease with total β-cell destruction. Notably, GSK126 did not significantly alter expression of the acinar marker; Amy2A, the ductal marker; Ck19 or the glucose-sensor; Txnip. Stimulation with GSK126 also increased expression of the proliferative marker Ki67. This was consistent with elevated expression of Sox9, a regulator of ductal progenitor cell expansion and differentiation.

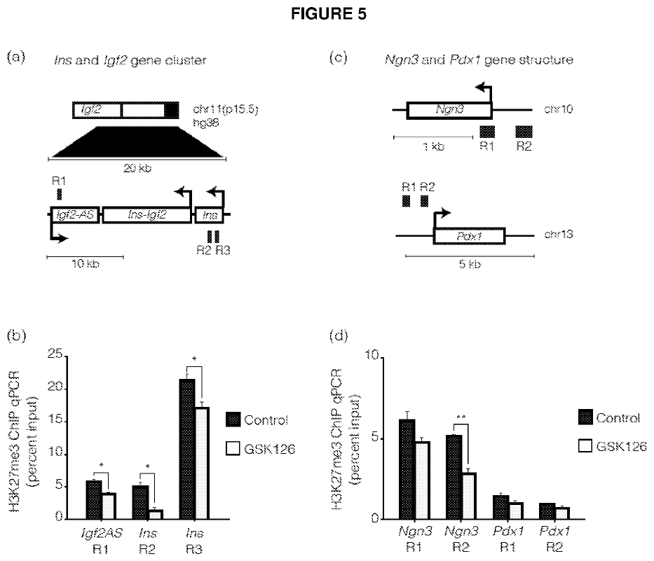

To determine whether H3K27me3 content on the Ins chromatin domain (Ins and Igf2AS) could influence default transcriptional suppression, chromatin immunoprecipitation (ChIP) experiments were employed from exocrine cells derived from non-diabetic donor (2) that were treated in the presence of (GSK126) or absence (vehicle control, DMSO) of the EZH2 inhibitor. Proteins were cross-linked to DNA and soluble chromatin fractionated by sonication. DNA was immunopurified using an antibody that specifically recognizes H3K27me3. DNA was assessed by qPCR using amplimers specifically designed to detect human Ins chromatin domain genes (FIG. 5a). Significant reduction in H3K27me3 content was observed at the Ins and Igf2AS promoter (R1-R3) regions in exocrine cells treated with GSK126 (FIG. 5b). H3K27me3 promoter content on Ngn3 and Pdx1 promoters was also examined (Figure Sc). In non-diabetic exocrine cells, H3K27me3 content on the Ngn3 promoter of R1 was marginally affected whereas R2 was significantly reduced in cells treated with GSK126 (FIG. 5d). Pdx1 DNA was recovered equally well from vehicle and GSK126-treated cells. These results may suggest the chromatin content of the Ins domain could be characterised by tight epigenetic control by H3K27me3 in human exocrine cells.

### DISCUSSION

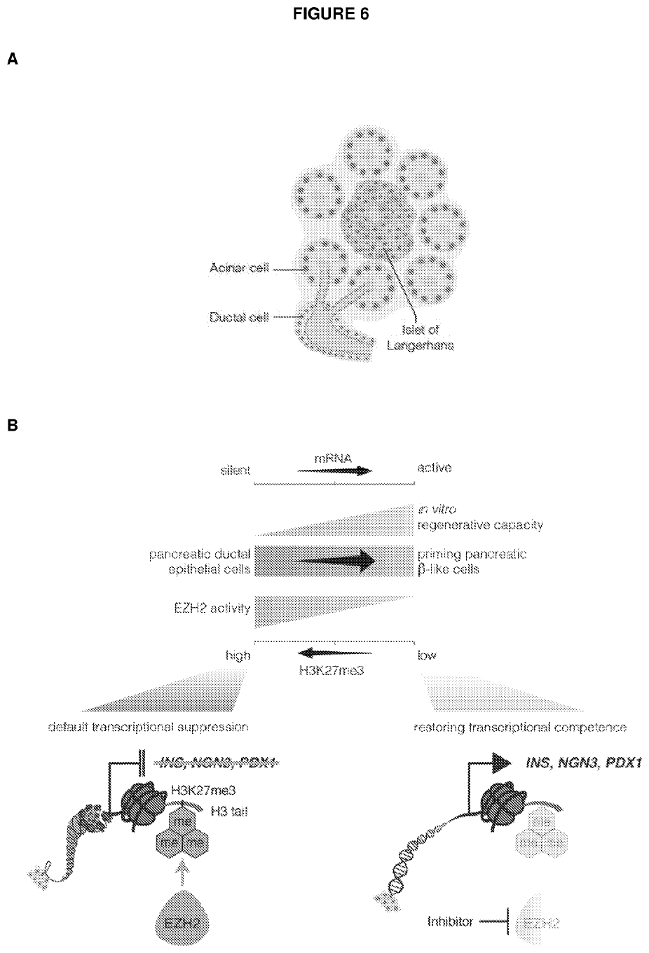

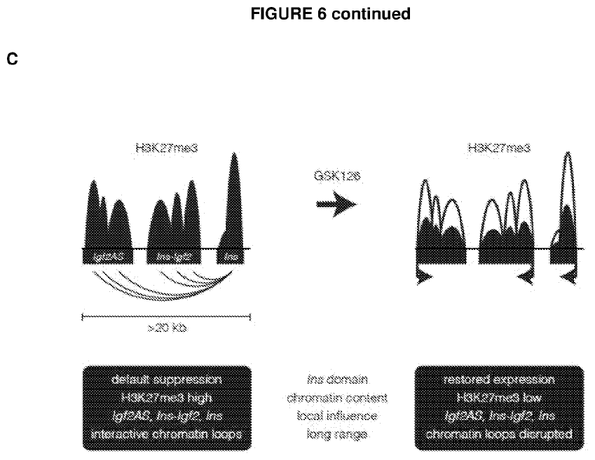

Taken together, the results may provide an indication that EZH2 inhibitors are useful as epigenetic compounds for β-cell regeneration. Not only are the data of direct clinical relevance, these studies are important for understanding mechanisms of gene regulation by pharmacological EZH2 inhibition. Because exocrine Pdx1, Ngn3, Sox9, Sox11, and β-cell specific markers Nkx6.1, MafA and Ins gene expression were shown to be restored ex vivo, it could be predicted that these changes may influence β-cell neogenesis and that GSK126 targets refractory EZH2 mediated suppression (FIG. 6a). MafA is expressed exclusively in β-cells and regulates the expression of genes involved in insulin synthesis and secretion. Nkx6.1 is a lineage allocation factor that drives progenitors toward a β-cell fate where its expression is restricted to 1-cells and found to be required for proper insulin secretion. Importantly, the results provide an indication that exocrine barrier accounts for the inability to restore core progenitor genes that influence ex vivo plasticity despite total β-cell destruction (FIG. 6b). The insulin gene is part of a large chromatin domain associated with histone modifications that function to regulate gene expression in human islets. The assembly of specialised nucleosomal structures on H3K27me3 chromatin implicates the refractory capacity of the Ins domain—to suppress transcription more effectively—than conventional or open chromatin of exocrine cells not treated with GSK126 (FIG. 6c).

The present results describe the first example of restoring exocrine transcription of the insulin gene, providing distinguishable evidence for β-cell regeneration and possibly neogenesis, which may have clinical implications for regenerative medicine within the context of T1 D. Furthermore, the results also demonstrate that human pancreatic progenitor cells retain their capacity to differentiate into neo-β-cells from the exocrine compartment that were derived from an insulin devoid T1 D donor. These results show that pharmacological alteration of cell fate decisions through the epigenetic induction of pancreatic progenitor genes holds promise for the treatment of T1 D by promoting ex vivo β-cell regeneration through assisted epigenetic lineage reprogramming.

### Example 2. Effect of Ursolic Acid and GSK126 on Human Pancreatic Ductal Cells

Human immortalised ductal cell line (Catalog #: T0018001, Addexbio) was cultured and assayed in Keratinocyte SFM (serum-free medium) (Catalog number 17005042, ThermoFisher Scientific, USA). Pharmacological inhibition of EZH2 and gene expression analysis were performed as described in Example 1. Human immortalised ductal cell were kept untreated or stimulated with ursolic acid (final concentration 1 μM, 2.5 μM or 5 μM) or GSK126 (final concentration 5, 10 or 20 μM).

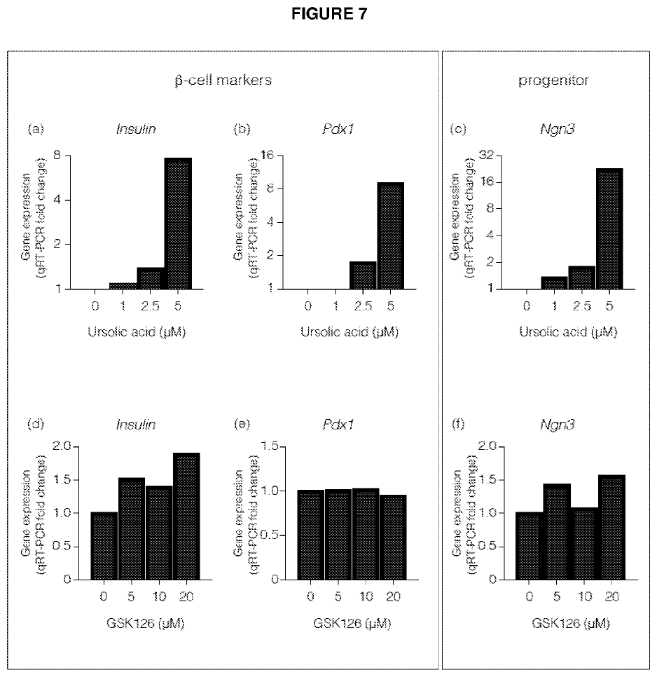

FIG. 7 illustrates the effects of pharmacological inhibition using ursolic acid or GSK126 on human immortalised ductal cells. Ursolic acid and GSK126 stimulated expression of Insulin and Pdx1, as well as the canonical master endocrine progenitor determinant Ngn3. These results may suggest EZH2 inhibition can restore exocrine multipotency.

### Example 3. Effect of Various EZH2 Inhibitors on Human and Marmoset Exocrine Cells

Exocrine cells from a non-diabetic human and a marmoset were cultured and assayed in Miami/CMRL medium. The human exocrine cells were sourced from the Islet Transplant Unit of St Vincent's Institute for Medical Research. The marmoset exocrine cells were sourced from the Australian Regenerative Medicine Institute (ARMI), Monash University. Miami media is based on CMRL 1066 media (Connaught Medical Research Lab) supplemented with: Insulin, Transferrin & Selenium (ITS), human serum, Vitamin E, sodium pyruvate, zinc and HEPES. Pharmacological inhibition of EZH2 and gene expression analysis were performed as described in Example 1. Human exocrine cells were untreated or stimulated with GSK126 (final concentration 10 μM), CPI-1205 (final concentration 5 μM), ursolic acid (final concentration 5 μM), GSK343 (final concentration 5 μM) or EPZ-6438 (final concentration 5 μM). Marmoset exocrine cells were untreated or stimulated with GSK126 (final concentration 10 μM) or ursolic acid (final concentration 5 μM).

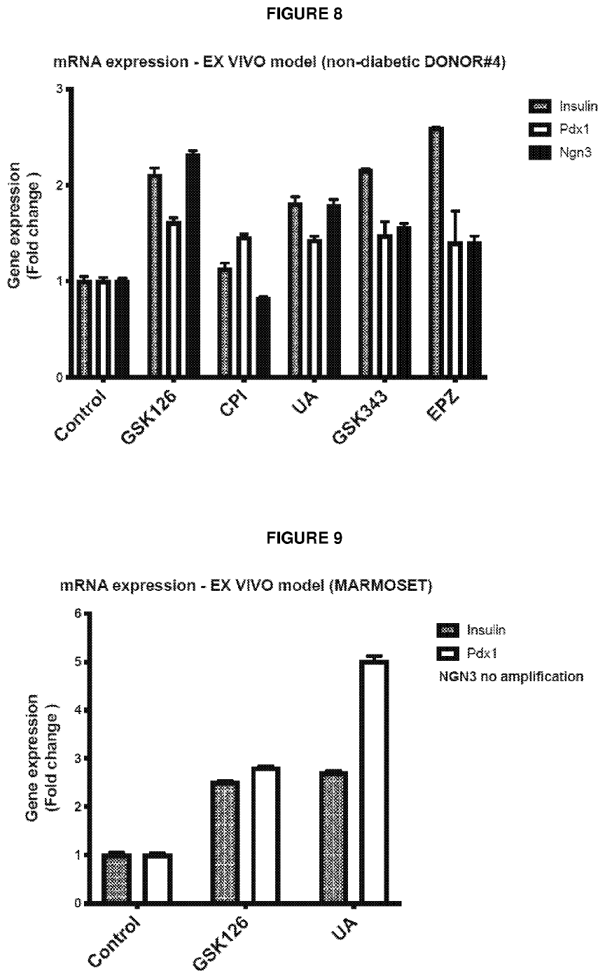

FIG. 8 illustrates the effects of pharmacological inhibition using GSK126, CPI-1205, ursolic acid, GSK343 or EPZ-6438 on human exocrine (acinar and ductal) cells from a non-diabetic donor. The EZH2 inhibitors stimulated expression of one, two or all of Insulin, Pdx1 and Ngn3. FIG. 9 illustrates the effects of pharmacological inhibition using GSK126 or ursolic acid on marmoset exocrine (acinar and ductal) cells. GSK126 and ursolic acid stimulated expression of Insulin and Pdx1. No amplification of Ngn3 was observed. These results may suggest EZH2 inhibition could restore exocrine multipotency in human and non-human primate exocrine cells.

### Example 4. Pharmacological Inhibition of Human EZH2 can Influence a Regenerative β-Cell Like Capacity with In Vitro Insulin Release in Pancreatic Ductal Cells

### Materials and Methods

### Cell Culture and EZH2 Inhibitor Stimulation

Human pancreatic ductal epithelial cells were purchased from AddexBio and cultured according to the recommended protocols. Cells were cultured in complete Keratinocyte Serum-Free Media (supplemented with human recombinant EGF, Bovine Pituitary Extract and Antibiotic-Antimycotic [Gibco]. All cell cultures were grown and maintained in a 37° C., 5% C₀₂ environment using a tissue culture incubator. Once cells reached 70-80% confluency, passaging was performed using 0.05% Trypsin EDTA (Sigma).

EZH2 inhibitors investigated in this study included the synthetically designed GSK-126 (S7061, SelleckChem), and EPZ-6438 (S7128, SelleckChem), as well as the naturally occurring compound, Triptolide (S3604, SelleckChem), which is known to display EZH2 inhibitor activity. Vehicle control was DMSO. Cells were treated over 2 main timepoints, with harvests occurring following 2, and 7 days.

For the 2-day time point, cells were seeded and left to adhere in plates for 24 hours. Treatment was initiated with the first dose made up in complete K-SFM. The second dose was delivered 24 hours later following a media change. For the longer period of 7 days, cells were initially seeded in 10 cm plates, with doses delivered on alternate days following the initial period of 24 hours. When the plate reached 90% confluency, cells were passaged using 0.05% Trypsin EDTA (Sigma) and re-seeded at a 1:2 dilution. 3 days prior to harvest, cells were passaged and seeded into cell culture plates depending on the application, with the final addition occurring like the 2-day treatment over 2 periods of 24 hours.

Quantitative real time PCR (qRT-PCR) was performed to examine differential gene expression using the following reaction mix: 5 uL Brilliant II SYBR® Green QPCR Master Mix (600903, Agilent Technologies), 2 uL nuclease-free water, 2 μL of template cDNA, and 0.5 uL of forward and reverse primer from oligoperfect designer (Thermo Fisher Scientific), detailed in Table 1. qRT-PCR cycles were carried out using Applied Biosystems 7500 Fast Real-Time PCR System, with each reaction consisting of a 3-minute hot start at 95° C., followed by 40 cycles of 5 seconds at 95° C., and 15 seconds at 60° C. Ct values of experimental genes were normalized to housekeeping gene H3F3A. Fold change of mRNA abundance was calculated by normalizing drug treated values to vehicular controls.

### RNA Extraction and Quantitative RT-PCR

TRIzol was used to extract total RNA from 5×105 cells seeded in 12 wells plates which were untreated (vehicle control DMSO) or incubated with EZH2 inhibitors for 2-, 7-days. RNA was isolated using the RNeasy Kit according to the manufacturer's directions. Following measurement of RNA concentration using a QIAxpert System, 1 μg of RNA was used for cDNA synthesis by a high-capacity cDNA Reverse Transcription Kit (Applied Biosystems). The resulting cDNA reaction mix was diluted 1:6 to make up the final template cDNA used subsequently.

Quantitative real time PR (qRT-PCR) was performed to examine differential gene expression using the following reaction mix: 5 uL Brilliant II SYBRR Green QPCR Master Mix (600903, Agilent Technologies), 2 uL nuclease-free water, 2 uL of template cDNA, and 0.5 uL of forward and reverse primer from oligoperfect designer (Thermo Fisher Scientific), detailed in Table 4. qRT-PCR cycles were carried out using Applied Biosystems 7500 Fast Real-Time PR System, with each reaction consisting of a 3-minute hot start at 9500, followed by 40 cycles of 5 seconds at 9500, and 15 seconds at 60GN. Ct values of experimental genes were normalized to housekeeping gene H3F3A. Fold change of mRNA abundance was calculated by normalizing drug treated values to vehicular controls.

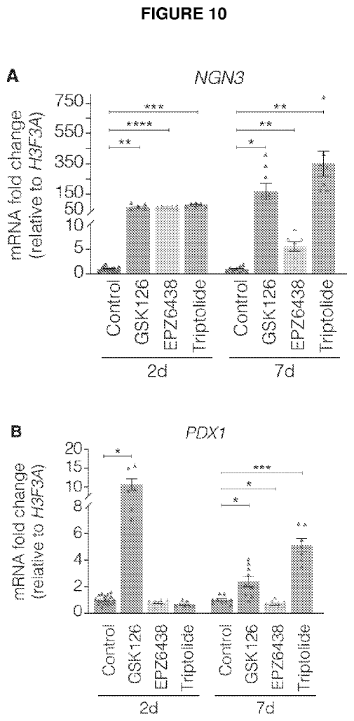

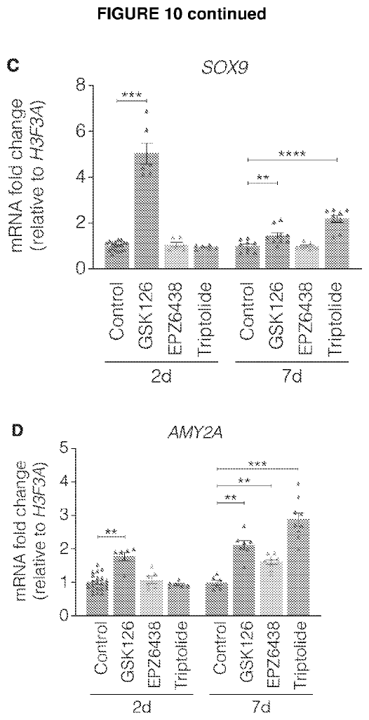

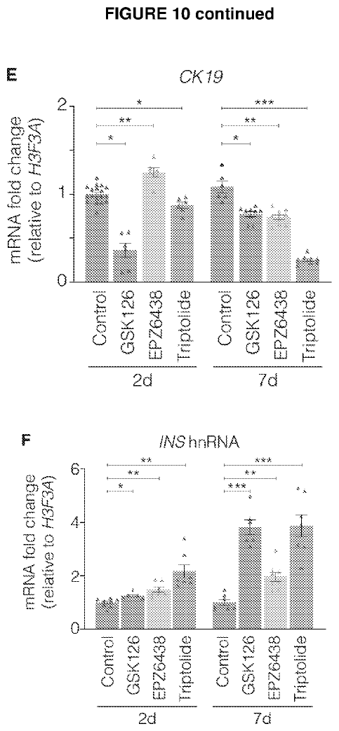

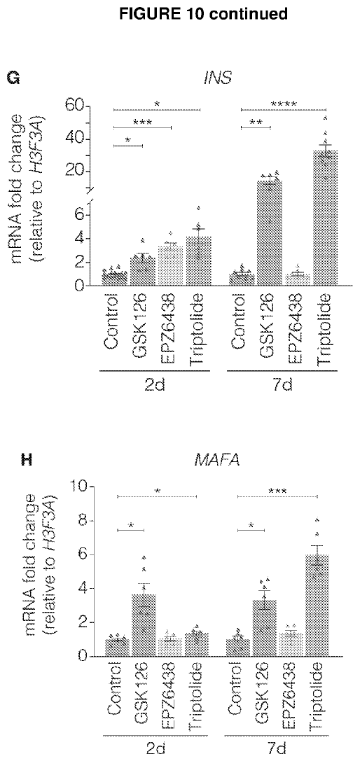

TABLE 4
Human primers for qRT-PCR

| Gene | Primer | Sequence | SEQ ID No. |
|---|---|---|---|
| H3F3A | human cDNA | ACAAAAGCCGCTCGCAAGAGTG | 55 |
| | Forward | | |
| | human cDNA | TTTCTCGCACCAGACGCTGGAA | 56 |
| | Reverse | | |
| INS | human cDNA | GCAGCCTTTGTGAACCAACAC | 57 (used in |
| | Forward | | FIG. 10G) |
| | human cDNA | CCCCGCACACTAGGTAGAGA | 58 (used in |
| | Reverse | | FIG. 10G) |
| NGN3 | human cDNA | CTAAGAGCGAGTTGGCACTGA | 59 (used in |
| | Forward | | FIG. 10A) |
| | human cDNA | GAGGTTGTGCATTCGATTGCG | 60 (used in |
| | Reverse | | FIG. 10A) |
| PDX1 | human cDNA | GAAGTCTACCAAAGCTCACGCG | 61 |
| | Forward | | |
| | human cDNA | GGAACTCCTTCTCCAGCTCTAG | 62 |
| | Reverse | | |
| SOX9 | human cDNA | AGGAAGCTCGCGGACCAGTAC | 63 |
| | Forward | | |
| | human cDNA | GGTGGTCCTTCTTGTGCTGCAC | 64 |
| | Reverse | | |
| CK19 | human cDNA | AGCTAGAGGTGAAGATCCGCGA | 65 |
| | Forward | | |
| | human cDNA | GCAGGACAATCCTGGAGTTCTC | 66 |
| | Reverse | | |
| AMY2A | human cDNA | GATAATGGGAGCAACCAAGTGGC | 67 |
| | Forward | | |
| | human cDNA | CAGTATGTGCCAGCAGGAAGAC | 68 |
| | Reverse | | |
| MAFA | human cDNA | GCTTCAGCAAGGAGGAGGTCAT | 69 |
| | Forward | | |
| | human cDNA | TCTGGAGTTGGCACTTCTCGCT | 70 |
| | Reverse | | |
| INS | human cDNA | GAGATGGGGAAGATGCTGGG | 71 |
| hnRNA | Forward | | |
| | human cDNA | GGAGGACACAGTCAGGGAGA | 72 |
| | Reverse | | |
| EZH2 | human cDNA | TCCTTTTCATGCAACACCCAACACT | 73 |
| | Forward | | |
| | human cDNA | TCCAAATGCTGGTAACACTGTGGTC | 74 |
| | Reverse | | |
| EP300 | human cDNA | GCAGTGTGCCAAACCAGATG | 75 |
| | Forward | | |
| | human cDNA | GGGTTTGCCGGGGTACAATA | 76 |
| | Reverse | | |
| KAT2 | human cDNA | ATTCTGCAGGGGCCGAGCCT | 77 |
| | Forward | | |
| | human cDNA | ATCACACGGAGCCGCTTGGC | 78 |
| | Reverse | | |
| BRG1 | human cDNA | GCTCATGGCTGAAGATGAGG | 79 |
| | Forward | | |
| | human cDNA | CAGGCGCTTGTCCTTCTTC | 80 |
| | Reverse | | |

Quantitative PCR Chromatin Immunoprecipitation (q-PCR ChIP)

Approximately 5×10⁶ cells were fixed in 1% formaldehyde for 10 minutes, with a further 10-minute incubation in 0.125M glycine to quench the cross-linking reaction. The fixed cell pellet was lysed following resuspension and homogenization in sodium dodecyl (lauryl) sulphate (SOS) lysis buffer (1% SOS, 10 mM EDTA, 50 mM Tris-HCl pH 8.1) with a protease inhibitor cocktail (Roche Diagnostics GmBH, Mannheim, Germany) included. Samples were incubated on ice for 5 minutes following which sonication was performed to shear chromatin between 200-600 bp. Sonicated chromatin was resuspended in ChIP Dilution Buffer (0.01% SOS, 1.1% Triton X-100, 1.2 mM EDTA, 16.7 mM Tris-HCl pH 8.0 and 167 mM NaCl). 20 μl of DynabeadsR Protein A (Invitrogen, Carlsbad, CA, USA) was added to each sample and pre-cleared. Overnight incubation at 4° C. with H3K27me3 or H3K9/14ac antibody was used for immunoprecipitation of chromatin, as previously described. Immunoprecipitates were collected by magnetic isolation and washed sequentially with low salt and high salt buffers. Immunoprecipitated DNA was then eluted from solution with 0.1 M NaHCO₃ containing 1% SIDS. Protein-DNA cross-links were reversed by incubation of samples in Proteinase K (Sigma, St. Louis, MO, USA) for 2 hours at 62° C. DNA was purified using a Qiagen MinElute column (Qiagen Inc., Valencia, CA, USA). H3K27me3 or H3K9/14ac content at the promoters of the INS-IGF2, NGN3 and PDX1 genes were assessed by qPCR using primers designed from the integrative ENCODE resource. ChIP primers are shown in Table 5.

TABLE 5
Human primers for q-PCR ChIP

| Gene | Primer | Sequence | SEQ ID No. |
|---|---|---|---|
| INS-IGF2 | PromR1 | GGGAACATAGAG | 81 |
| | Forward | AAAGAGGTCTCA | |
| | PromR1 | AATTAATCTCAG | 82 |
| | Reverse | CTTCCCCCTAAC | |
| NGN3 | PromR1 | TTGCTCCTAGCC | 83 |
| | Forward | TATCTTTCCTTA | |
| | PromR1 | CTTTAGAATTCC | 84 |
| | Reverse | TGGACCCTTCTC | |
| PDX1 | PromR1 | ACGTTTCTGCAA | 85 |
| | Forward | AGCTGTCTAGTT | |
| | PromR1 | GGCTTCAAACCA | 86 |
| | Reverse | TTCAGTAACTTC | |

### Protein Blot

Histone proteins were extracted from 1×10⁶ cells per sample. Acid extraction of nuclear proteins and immunoblotting was performed as previously described (Kaipananickal H, et al. Targeting treatment refactory NET by EZH2 inhibition in postural tachycardia syncrome. Circ Res, 126(8) (2020)). Protein content of samples were incubated using Bradford's Reagent (Sigma), with standard concentrations of BSA used to determine concentration. 1 ug of protein per sample was run on a 4-12% gel (Nu-Page, Invitrogen) before transfer to a PVDF membrane. Membranes (Immobilon-FL; Millipore) were incubated in primary antibodyagainst H3 (1B1B2, CST), H3K27ac (ab4729, Abcam), EZH2 (#4905, CST), β-actin (ab8226, Abcam) and H3K27me3 (07-229, Millipore) overnight (dilutions listed in table 3). Membranes were incubated in secondary antibody and imaged using LiCoR Odyssey infrared system. Image studio was used to quantify the protein bands with total H3 or R-actin as a loading control.

### Immunofluorescence

20×10⁴ cells were seeded on 15 mm coverslips in 24 well plates and treated with EZH2 inhibitors or vehicle control over 2, or 7 days. Cells were fixed in 4% PFA. 0.1% Triton X diluted in PBS was used to permeabilize cells for 10 minutes, followed by blocking in a solution of 0.2% gelatin, 2.5% bovine serum albumin made up in PBS (PBG). Primary antibodies against CK19 (HPA002465 Sigma-Aldrich), and INS (A0564, DAKO) were made up in PBG according to the dilutions listed in Table 6 and incubated overnight at 4° C. Coverslips were washed and incubated with secondary antibodies against rabbit (Alexa Fluor 488), and goat (IRDye® 680CW) (dilutions in Table 6) for 1 hour at room temperature. Cells were then washed and incubated with 4′,6-diamidino-2-phenylindole (DAPI) as a nuclear counterstain (at a 1:100 dilution from a 10 μg/ml stock; D8417 Sigma-Aldrich) for 10 minutes prior to mounting using Prolong Gold Anti-Fade mountant with DAPI (ThermoFisher). Slides were viewed and images were obtained from EVOS (ThermoFisher) using the TagBFP, Cy5, and GFP filters. Images were processed using Image J.

TABLE 6
Antibody dilutions for western blot and immunofluorescent
staining of human pancreatic ductal epithelial cells

| Antibody | Dilution |
|---|---|
| Rabbit H3K27me3 | 1:2500 |
| Mouse Total H3 | 1:1000 |
| Rabbit H3K27ac | 1:1000 |
| Mouse β-actin | 1:10000 |
| Rabbit EZH2 | 1:1000 |
| IRDye ® 680CW Goat Anti-Rabbit | 1:10000 |
| IRDye ® 800CW Goat Anti-Mouse | 1:10000 |
| Rabbit Anti-CK19 | 1:200 |
| Guinea Pig Anti-Insulin | 1:250 |
| Alexa Fluor 488 Donkey Anti-Rabbit | 1:1000 |
| IRDye ® 680CW Donkey Anti-Guinea Pig | 1:1000 |

### Glucose Stimulated Insulin Secretion Assay

5×10⁵ cells were seeded in 12-well plates and allowed to adhere for 24 hours. Given the relatively high concentration of insulin in KSFM, cells were washed and cultured with CMRL-complete (CMRL 1066supplemented with Antibiotic-Antimycotic [Gibco], and Glutamax [Gibco]) to reduce the background insulin concentration of the ELISA. Cells stimulated with EZH2 inhibitors or vehicle control (DMSO) for 2, or 7 days, following which they were washed with 2.8 mM glucose Krebs Buffer Solution (25 mM HEPES, 115 mM sodium chloride, 24 mM sodium hydrogen carbonate, 5 mM potassium chloride, 1 mM magnesium chloride heptahydrate, 0.1% bovine serum albumin, and 2.5 mM calcium chloride dihydrate made up in deionized water) two times, and incubated for 1 hour to obtain the low glucose (basal) insulin secretion. Next, cells were cultured in 28 mM (High) glucose Krebs Buffer solution and incubated for 1 hour to obtain the glucose stimulated insulin secretion. Supernatant was collected and Ultrasensitive Insulin ELISA (Mercodia) was used to determine the concentration of insulin according to manufacturer's instructions. Fold change of insulin secreted by cells were calculated by adjusting to cells stimulated with EZH2 inhibitors for insulin concentrations compared to control.

### Results

To examine whether pharmacological EZH2 inhibition could influence the terminal differentiated status, adult human pancreatic ductal epithelial cells were stimulated with GSK-126, EPZ6438 and Triptolide over 2- and 7-days (FIG. 10). Genes investigated included those important during endocrine development, as well as the expression of genes involved in retention of identity post-development for both ductal and β-cells. Culture of pancreatic ductal cells with GSK-126, EPZ6438 and triptolide for 2 days significantly elevated NGN3 mRNA levels (FIG. 10a). Further increases were observed at 7 days with GSK-126 and Triptolide. Alpha-amylase (AMY2A) transcripts were increased significantly following 2 days of GSK-126 stimulation while all EZH2 inhibitors significantly elevated AMY2A mRNA levels (FIG. 10d). There was a decrease in the cytoplasmic CK19 following 2 days of treatment (FIG. 10e) showing statistically significant decreases upon stimulation with GSK-126, and Triptolide. Although EPZ-6438 demonstrated an increase at the 2-day time point, by day 7 CK19 expression levels had decreased significantly to match the reduction seen with the other compounds (FIG. 10e). Inhibition of EZH2 in pancreatic ductal cells stimulated a statistically significant increase in insulin transcripts of both the unprocessed heterogenous nuclear form (FIG. 10f) as well as the mature mRNA (FIG. 10g). The transcriptional alterations demonstrated similar trends, with elevations following 2 days of treatment when stimulated with GSK-126, EPZ-6438, and Triptolide. The increase in gene expression was maintained at 7 days with even further increases for Triptolide, as well as GSK-126. V-maf musculoaponeurotic fibrosarcoma oncogene homolog A, or MAFA mRNA levels increased in a statistically significant manner at the 2-day point only upon treatment with GSK-126 and Triptolide (FIG. 10g). Whilst the fold change compared to vehicle treatment was stable for GSK-126 treatment at 7 days, EPZ-6438 similarly began to induce a statistically significant increase in MAFA transcript.

### Example 5. H3K27Me3 Content is Reduced in Endocrine Genes Following EZH2 Inhibition

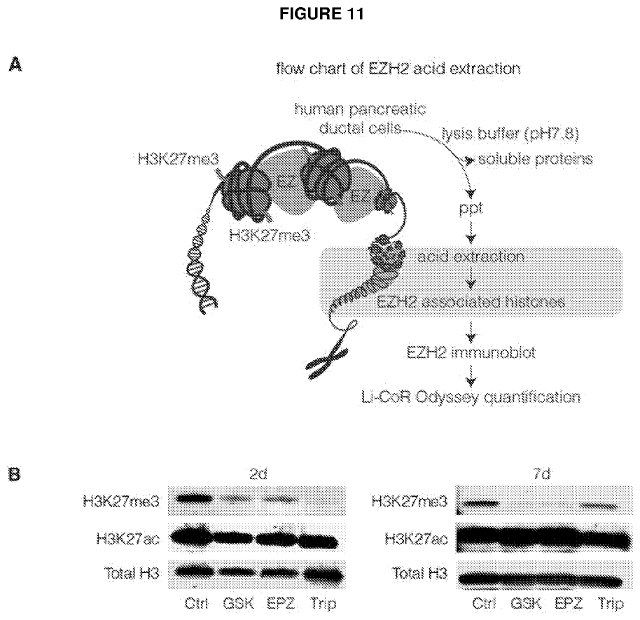

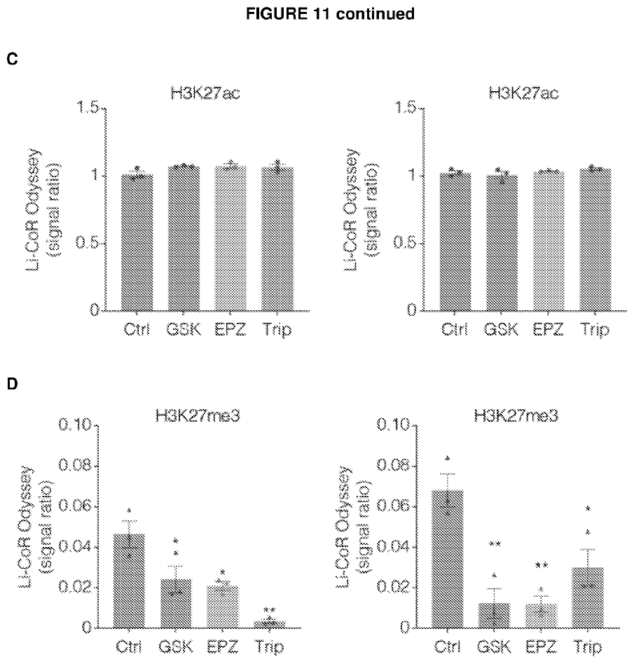

To confirm that the EZH2 inhibitors were working to reduce EZH2 activity within the nuclear chromatin, histone proteins were acid extracted from pancreatic ductal cells and analysed for H3K27me3 and H3K27ac content relative to total H3 extracted (FIG. 11a). There was a lack of change in H3K27ac between EZH2i treatments and controls (FIGS. 11b and c). Following 2 days, the drugs significantly reduced H3K27me3 in the pancreatic ductal cells when compared to the vehicle treated controls (FIGS. 11b and d). Prolonged stimulation over 7 days further decreased H3K27me3 with GSK126 and EPZ6438 (FIGS. 11b and d).

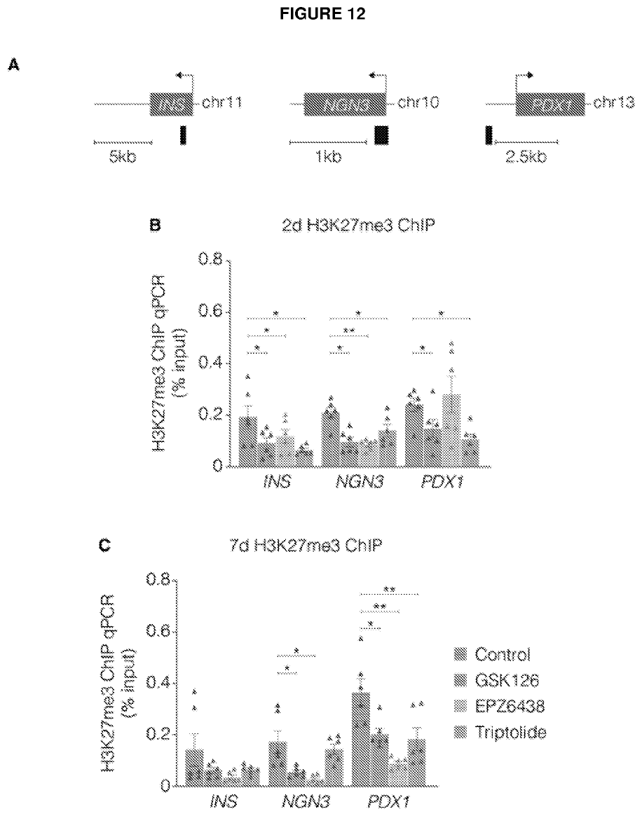

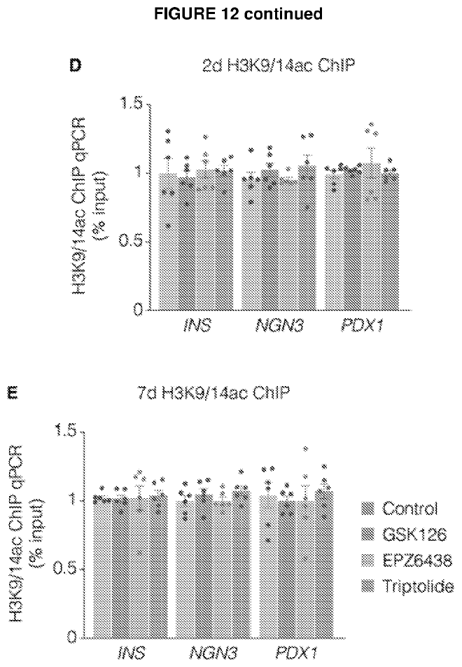

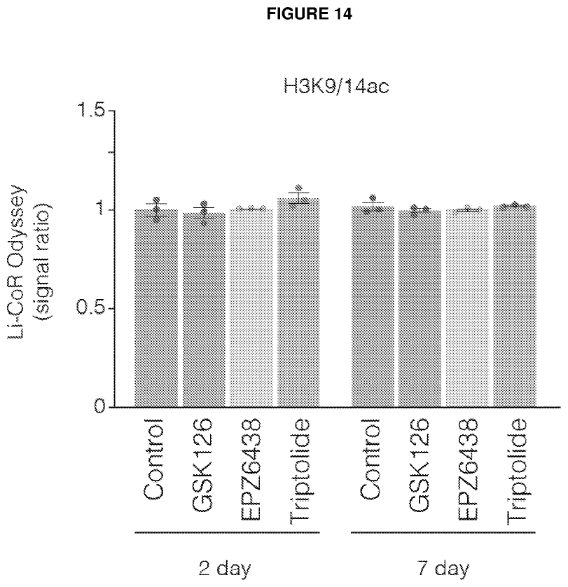

Given that EZH2 is involved in writing H3K27me3 which is associated with gene repression, assessment of whether gene expression as a result of treatment with EZH2 inhibitors could influence H3K27me3 content of related genes. H3K27me3 associated chromatin was immunopurified (ChIP) from pancreatic ductal cells. qPCR was used to assess H3K27me3 content using primers specifically designed to detect the promoter regions of INS, endocrine master regulator NGN3, and β-cell marker PDX1 (FIG. 12a). ChIP revealed GSK-126 significantly reduced the H3K27me3 content of chromatin associated with the INS promoter domain, as well as NGN3 and PDX1 (FIG. 12b). Additionally, these results were also demonstrated with Triptolide. EPZ-6438 reduced H3K27me3 content of PDX1 at 7 days (FIG. 12c). Furthermore, a lack of change in the H3K9/14 acetylation (H3K9/14ac) demonstrates the specificity of the EZH2i in modulating the trimethylation signal of HPDE cells which is further correlated with a lack of change as assessed by western blots (FIG. 14).

### Example 6. EZH2 Inhibition Stimulates Insulin Expression in Human Pancreatic Ductal Epithelial Cells

To determine whether increased INS and CK19 gene expression were indicative of a functional synthesis of the protein, cells stimulated with EZH2 inhibitors for 2 and 7 days were stained using immunofluorescence for INS and CK19 with DAPI serving as a control nuclear stain (Data not shown). As expected, the nuclear stain DAPI was imaged in cells that were stained. All cells stained were positive for CK19 indicating their ductal cell identity. Importantly, a population of pancreatic ductal cells were positive for insulin, which were not present in the DMSO controls as indicated by a lack of signal in the control cells when imaged for insulin. An average of 3 in 20,000 cells were observed in both GSK-126 and EPZ-6438 stimulated cells. Following 7 days, there was an overall increase in the numbers of insulin-positive cells, averaging 7 in 20,000 cells per treatment (Data not shown). Likewise, the cells stained positively for DAPI and CK19, confirming their identity as pancreatic ductal cells.

### Example 7. Human Pancreatic Ductal Epithelial Cells are Capable of Releasing Insulin

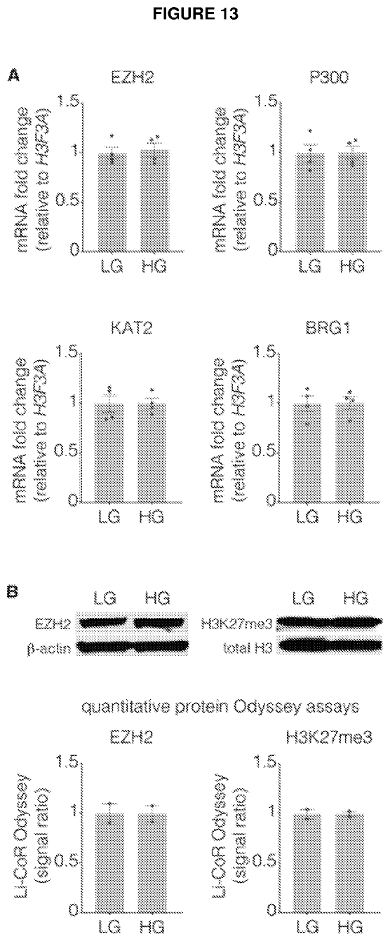

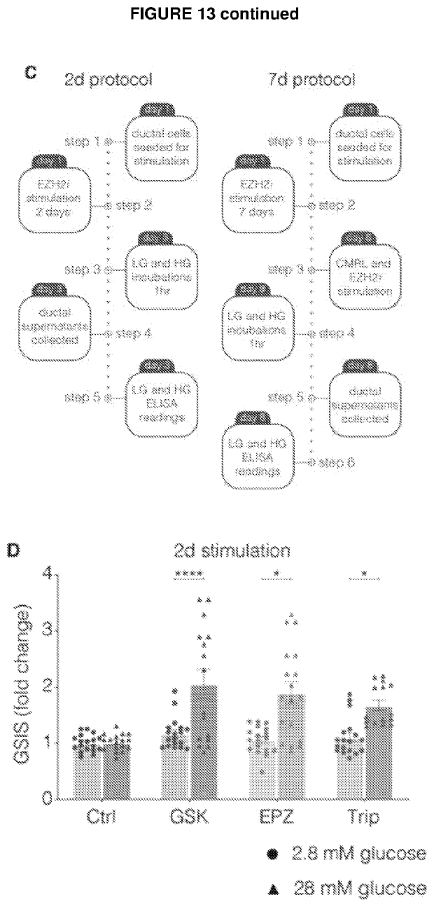

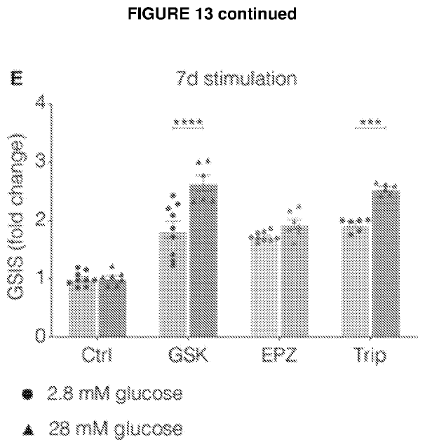

Since insulin was detected by pharmacological EZH2 inhibition we assessed glucose stimulated insulin secretion (GSIS). To confirm specificity of the assay, basal and stimulating levels of EZH2 and H3K27me3 were established by RT-qPCR (FIG. 13a), as well as western blot (FIG. 13b). Additionally, mRNA levels of the histone acetyltransferases P300, KAT2, and chromatin remodeler BRG1 were unchanged subsequent one hour of incubation in high glucose. There was no change in EZH2 expression, or protein levels as demonstrated by the lack of change in trimethylation following exposure to high glucose, thus confirming that any subsequent alterations in insulin concentration were due to EZH2i stimulation. The GSIS assay was performed following EZH2i stimulation (FIG. 13c) to examine if those insulin-producing pancreatic ductal cells were functional in their capacity to produce insulin under one hour of incubation in basal (2.8 mM glucose) or stimulating (28 mM glucose) conditions. Following incubation in high-glucose media, significant elevations in the insulin concentration was observed from human pancreatic ductal epithelial cells stimulated with GSK-126, EPZ6438 and Triptolide (FIG. 13d) which was not observed in the control DMSO treated cells. Release of insulin was also maintained at 7 days with GSK126 and Triptolide (FIG. 13e).

1. A method of producing an insulin-producing cell from a pancreatic exocrine cell, the method comprising:
contacting a pancreatic exocrine cell, or a cell population comprising a pancreatic exocrine cell, with an inhibitor of EZH2 for a sufficient time and under conditions to allow generation of an insulin-producing cell from the pancreatic exocrine cell,
thereby producing an insulin-producing cell, or a cell population comprising an insulin-producing cell.

2. The method according to claim 1, wherein the insulin-producing cell is characterised by increased expression of one or more pancreatic progenitor markers, relative to a pancreatic exocrine cell that has not been contacted with the inhibitor of EZH2.

3. The method according to claim 2, wherein the one or more pancreatic progenitor markers are selected from Pdx1, Ngn3, Sox9 and Sox11.

4. The method according to any one of claims 1 to 3, wherein the insulin-producing cell is characterised by increased expression of one or more β-cell markers, relative to a pancreatic exocrine cell that has not been contacted with the inhibitor of EZH2.

5. The method according to claim 4, wherein the one or more β-cell markers are selected from Nkx6.1, MafA, Ins hnRNA and Ins.

6. The method according to any one of claims 1 to 5, wherein the insulin-producing cell is characterised by increased expression of one or more proliferation genes, relative to a pancreatic exocrine cell that has not been contacted with the inhibitor of EZH2.

7. The method according to claim 6, wherein the one or more proliferation genes comprise Ki67.

8. The method according to any one of claims 1 to 7, wherein the insulin-producing cell is characterised by increased expression of one or more of Pdx1, Ngn3, Sox9, Sox11, Nkx6.1, MafA, Ins, Ins hnRNA and Ki67, relative to a pancreatic exocrine cell that has not been contacted with the inhibitor of EZH2.

9. The method according to any one of claims 1 to 8, wherein the insulin-producing cell is characterised by no change in expression of one or more of the following, relative to a pancreatic exocrine cell that has not been contacted with the inhibitor of EZH2: one or more ductal markers; one or more acinar markers; and one or more glucose-sensor markers.

10. The method according to claim 9, wherein one or more of the following apply: the one or more ductal markers comprise Ck19; the one or more acinar markers comprise Amy2A; and the one or more glucose-sensor markers comprise Txnip.

11. The method according to any one of claims 1 to 10, wherein the insulin-producing cell is capable of secreting insulin upon stimulation with glucose.

12. The method according to claim 11, wherein the insulin-producing cell is capable of repeatedly performing glucose stimulated insulin secretion (GSIS).

13. The method according to any one of claims 1 to 12, wherein the insulin-producing cell is a β-cell or a β-cell-like cell.

14. The method according to any one of claims 1 to 13, wherein the pancreatic exocrine cell is an acinar cell or a ductal cell.

15. The method according to claim 14, wherein the pancreatic exocrine cell is a ductal cell.

16. The method according to any one of claims 1 to 13, wherein the cell population comprising a pancreatic exocrine cell comprises or consists of one or both of acinar cells and ductal cells.

17. The method according to claim 16, wherein the cell population consists of ductal cells.

18. The method according to any one of claim 1 to 17, wherein the pancreatic exocrine cell, or the cell population comprising the pancreatic exocrine cell, is obtained from pancreatic tissue.

19. The method according to claim 18, wherein the pancreatic tissue comprises or consists of pancreatic exocrine cells.

20. The method according to claim 18 or claim 19, wherein the pancreatic tissue comprises or consists of mature terminally differentiated pancreatic cells.

21. The method according to any one of claims 1 to 20, wherein the inhibitor of EZH2 directly inhibits the enzymatic activity of EZH2.

22. The method according to claim 21, wherein the inhibitor of EZH2 occupies the site for co-substrate S-adenosylmethionine (SAM) in the binding pocket of EZH2.

23. The method according to claim 21 or claim 22, wherein the inhibitor of EZH2 competes with SAM for binding to EZH2.

24. The method according to any one of claims 1 to 23, wherein the inhibitor of EZH2 is a compound having the following structure: 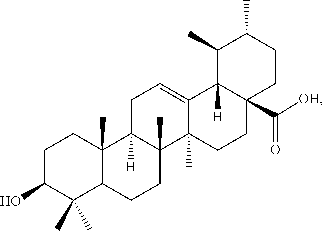
or a stereoisomer, a tautomer, a hydrate, a solvate, or a salt thereof, in particular a pharmaceutically acceptable salt, or a mixture of same.

25. The method according to any one of claims 1 to 23, wherein the inhibitor of EZH2 is a compound of formula (I): 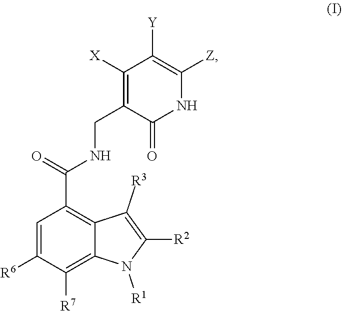
wherein:
X and Z are selected independently from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁—C)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halo, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, NR^{a}R^{b}, NR^{a}C(O)R^{b}, NR^{a}C(O)NR^{a}R^{b}, —NR^{a}C(O)OR^{a}, —NR^{a}SO₂R^{b}, NR^{a}SO₂NR^{a}R^{b}, NR^{a}NR^{a}R^{b}, NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};
Y is H or halo;
R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, —CONR^{a}NR^{a}R^{b};
R² is hydrogen, (C₁-C₈)alkyl, trifluoromethyl, alkoxy, or halo, in which said (C₁-C₈)alkyl maybe substituted with one to two groups selected from: amino, and (C₁-C₃)alkylamino;
R⁷ is hydrogen, (C₁-C₃)alkyl, or alkoxy; R³ is hydrogen, (C₁-C₈)alkyl, cyano, trifluoromethyl, —NR^{a}R^{b}, or halo;
R⁶ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-Cg)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, NR^{a}C(O)NR^{a}R^{b}, NR^{a}C(O)OR^{a}, NR^{a}SO₂R^{b}, NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, NR^{a}NR^{a}C(O)OR^{a}, —OR^{a}, —OC(O)R^{a}, —OC(O)NR^{a}R^{b};
wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, NR^{a}C(O)R^{b}, —NR^{a}C(O)NR^{a}R^{b}, —NR^{a}C(O)OR^{a}, NR^{a}SO₂R^{b}, NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, NR^{a}R^{b}, NR^{a}C(O)R^{b}, NR^{a}C(O)NR^{a}R^{b}, NR^{a}C(O)OR^{a}, —NR^{a}SO₂R^{b}, NR^{a}SO₂NR^{a}R^{b}, —OR^{a}, —OC(O)R^{a}, and —OC(O)NR^{a}R^{b};
each R^{o} is independently (C₁-C₄)alkylamino, —NR^{a}SO₂R^{b}, —SOR^{a}, —SO₂R^{a}, —NR^{a}C(O)OR^{a}, —NR^{a}R^{b}, or —CO₂R^{a};
R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl)((C₁-C₄)alkyl);
or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;
or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;
or a stereoisomer, a tautomer, a hydrate, a solvate, or a salt thereof, in particular a pharmaceutically acceptable salt, or a mixture of same.

26. The method according to claim 25, wherein the compound of formula (I) has the following structure: 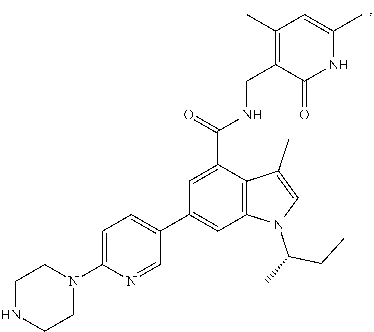 [SMILES: CC[C@H](C)n1cc(C)c2c(C(=O)NCc3c(C)cc(C)[nH]c3=O)cc(-c3ccc(N4CCNCC4)nc3)cc21]
or a stereoisomer, a tautomer, a hydrate, a solvate, or a salt thereof, in particular a pharmaceutically acceptable salt, or a mixture of same.

27. The method according to any one of claims 1 to 23, wherein the inhibitor of EZH2 is a compound having the following structure: 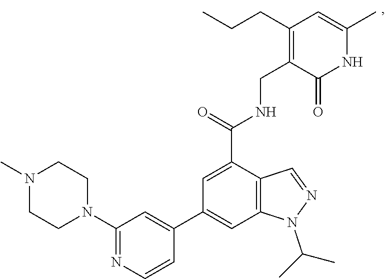
or a stereoisomer, a tautomer, a hydrate, a solvate, or a salt thereof, in particular a pharmaceutically acceptable salt, or a mixture of same.

28. The method according to any one of claims 1 to 23, wherein the inhibitor of EZH2 is a compound having the following structure: 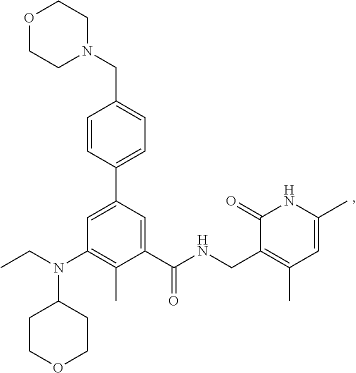 [SMILES: CCN(c1cc(-c2ccc(CN3CCOCC3)cc2)cc(C(=O)NCc2c(C)cc(C)[nH]c2=O)c1C)C1CCOCC1]
or a stereoisomer, a tautomer, a hydrate, a solvate, or a salt thereof, in particular a pharmaceutically acceptable salt, or a mixture of same.

29. The method according to any one of claims 1 to 23, wherein the inhibitor of EZH2 is a compound having the following structure: 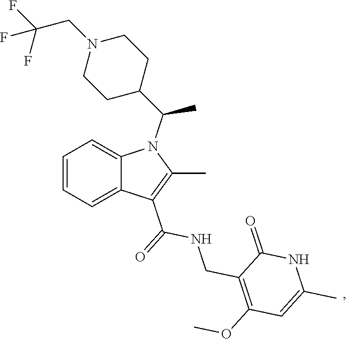 [SMILES: COc1cc(C)[nH]c(=O)c1CNC(=O)c1c(C)n([C@H](C)C2CCN(CC(F)(F)F)CC2)c2ccccc12]
or a stereoisomer, a tautomer, a hydrate, a solvate, or a salt thereof, in particular a pharmaceutically acceptable salt, or a mixture of same.

30. The method according to any one of claims 1 to 23, wherein the inhibitor of EZH2 is a compound having the following structure: 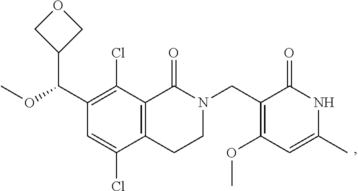
or a stereoisomer, a tautomer, a hydrate, a solvate, or a salt thereof, in particular a pharmaceutically acceptable salt, or a mixture of same.

31. The method according to any one of claims 1 to 23, wherein the inhibitor of EZH2 is a compound having the following structure: 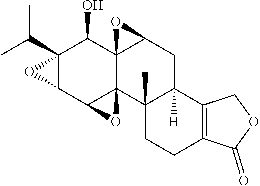
or a stereoisomer, a tautomer, a hydrate, a solvate, or a salt thereof, in particular a pharmaceutically acceptable salt, or a mixture of same.

32. The method according to any one of claims 1 to 23, wherein the inhibitor of EZH2 is a compound having the following structure: 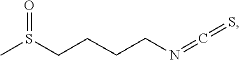
or a stereoisomer, a tautomer, a hydrate, a solvate, or a salt thereof, in particular a pharmaceutically acceptable salt, or a mixture of same.

33. The method according to any one of claims 1 to 32, wherein the method further comprises a step of administering the insulin-producing cell, or the cell population comprising the insulin-producing cell, to an individual.

34. The method according to claim 33, wherein the pancreatic exocrine cell from which the insulin-producing cell is generated is obtained from the individual.

35. The method according to claim 33 or claim 34, wherein the individual has diabetes mellitus or is pre-diabetic.

36. The method according to claim 35, wherein the diabetes mellitus is selected from type 1 diabetes, type 2 diabetes and gestational diabetes.

37. An insulin-producing cell, or a cell population comprising an insulin-producing cell, produced by the method according to any one of claims 1 to 36.

38. Use of an inhibitor of EZH2 in the manufacture of a composition for producing an insulin-producing cell from a pancreatic exocrine cell.

39. An inhibitor of EZH2 for use in producing an insulin-producing cell from a pancreatic exocrine cell.

40. A method for preventing or treating a disease involving dysfunctional insulin production, the method comprising:
administering a cell population comprising an insulin-producing cell according to claim 37 or produced by the method according to any one of claims 1 to 36, to an individual in need thereof,
thereby preventing or treating the disease.

41. The method according to claim 40, wherein the disease involving dysfunctional insulin production is selected from diabetes mellitus and pre-diabetes.

42. The method according to claim 41, wherein the diabetes mellitus is selected from type 1 diabetes, type 2 diabetes and gestational diabetes.

43. The method according to any one of claims 40 to 42, wherein the pancreatic exocrine cell from which the insulin-producing cell of the cell population is generated is from pancreatic tissue obtained from the individual.

44. The method according to any one of claims 40 to 43, wherein the cell population comprising the insulin-producing cell is delivered to the pancreas of the individual.

45. Use of an insulin-producing cell or a cell population comprising an insulin-producing cell according to claim 37 or produced by the method according to any one of claims 1 to 36, in the manufacture of a composition for preventing or treating a disease involving dysfunctional insulin production in an individual.

46. An insulin-producing cell or a cell population comprising an insulin-producing cell according to claim 37 or produced by the method according to any one of claims 1 to 36, for use in preventing or treating a disease involving dysfunctional insulin production in an individual.

47. A method for preventing or treating a disease involving dysfunctional insulin production, the method comprising:
administering an inhibitor of EZH2 to an individual in need thereof such that the inhibitor of EZH2 contacts a pancreatic exocrine cell of the individual for a sufficient time and under conditions to allow generation of an insulin-producing cell from the pancreatic exocrine cell,
thereby preventing or treating the disease in the individual.

48. The method according to claim 47, wherein the disease involving dysfunctional insulin production is selected from diabetes mellitus and pre-diabetes.

49. The method according to claim 48, wherein the diabetes mellitus is selected from type 1 diabetes, type 2 diabetes and gestational diabetes.

50. The method according to any one of claims 47 to 49, wherein the inhibitor of EZH2 is as defined in any one of claims 21 to 32.

51. Use of an inhibitor of EZH2 in the manufacture of a composition for preventing or treating a disease involving dysfunctional insulin production in an individual, wherein the inhibitor of EZH2 contacts a pancreatic exocrine cell of the individual for a sufficient time and under conditions to allow generation of an insulin-producing cell from the pancreatic exocrine cell.

52. An inhibitor of EZH2 for use in preventing or treating a disease involving dysfunctional insulin production in an individual, wherein the inhibitor of EZH2 contacts a pancreatic exocrine cell of the individual for a sufficient time and under conditions to allow generation of an insulin-producing cell from the pancreatic exocrine cell.
